(19) United States
(12) Patent Application Publication
(10) Pub. No.: US 2004/0138161 A1
(43) Pub. Date: Jul. 15, 2004

(54) Methods of modulating proliferative conditions

(76) Inventors: Bruno Amati, Milan (IT); Paula C. Fernandez Vogel, Aarau (CH); Scott R. Frank, Cambridge, MA (US)

Correspondence Address: DNAX RESEARCH, INC., LEGAL DEPARTMENT, 901 CALIFORNIA AVENUE, PALO ALTO, CA, 94304, US

(21) Appl. No.: 10/625,486

(22) Filed: Jul. 22, 2003

Related U.S. Application Data
(60) Provisional application No. 60/398,088, filed on Jul. 24, 2002.

(51) Int. Cl. A61K 48/00; A61K 39/395; C12Q 1/68; G01N 33/574

(52) U.S. Cl. 514/44; 424/155.1; 514/12; 435/6; 435/7.23

(57) ABSTRACT

A novel group of Myc-targets from the human genome is provided. Also provided are methods of making and using reagents for modulating activity of Myc-target gene products, and for the diagnosis and treatment of proliferative conditions that are regulated by Myc.

This application claims benefit from U.S. Provisional Patent Application No. 60/398,088, filed Jul. 24, 2002.

### FIELD OF THE INVENTION

The present invention discloses methods for the diagnosis and treatment of proliferative conditions, e.g., cancer. In particular, it provides identification of a group of Myc-binding genes, and methods of using agonists or antagonists that modulate the activities of these genes and their gene products.

### BACKGROUND OF THE INVENTION

Cancer of the gastrointestinal tract, pancreas, liver, prostate, breast, and the leukemias, are among the most frequent types of cancer (Tichopoulos, et al. (1997) in Epidemiology of Cancer in Cancer: Principles and Practice of Oncology, Fifth Ed., ed. by DeVita, et al., Lippincott-Raven Publishers, Phila., PA, pp. 231-257). Cancer develops in stages from normal cells to benign lesions, to malignant tumors such as carcinomas, and finally to invasive metastatic disease. Alterations in gene structure orgene expression appear to be responsible for the progression of cancer.

Nearly all cells in the body grow and divide, with well regulated periods of quiescence. These quiescent periods are markedly decreased or improperly regulated in cancer cells. Cell proliferation, i.e., the rate of cell division, is controlled by genes that regulate the rate of cell growth, division, and quiescence. In addition to increased cell proliferation, cancer is distinguished by changes in the regulation of genes that control angiogenesis and metastasis. Cells divide or remain quiescent as a result of certain proteins that function to regulate intracellular messages. Some of these proteins bind to DNA and regulate gene activity by binding DNA, while others are membrane-bound or remain free in solution. Examples of signaling proteins that bind to DNA include, e.g., Myc, Ras, Jun, and Fos. Examples of signaling proteins that do not bind to DNA include, e.g., cyclins and certain protein kinases.

Myc gene is closely associated with the etiology of cancer, as mutations or changes in intracellular levels of Myc occur in various cancers. Myc protein has been implicated in the regulation of a number of genes. The identification of which of these putative Myc-regulated genes are important to cancer has been difficult. The present invention provides a solution to this problem by disclosing a group of genes comprising regulatory regions that bind Myc, in vivo.

### SUMMARY OF THE INVENTION

The present invention is based, in part, upon the discovery of a group of Myc-binding genes that can affect cell proliferation and cancer.

The invention provides a method of regulating cell proliferation comprising modulating the activity of a gene or polypeptide of Table 2, the above method wherein the gene is positive for Myc binding in a chromatin immunoprecipitation (ChIP) assay, the above method wherein the modulating is inhibiting or activating; and the above method wherein the cell proliferation is oncogenic.

Also provided is a method of regulating cell proliferation comprising modulating the activity of gene or polypeptide of Table 2, wherein the modulating is by a binding composition, or wherein the binding composition comprises an antigen-binding site of an antibody, a soluble receptor, a nucleic acid, or a small molecule, or wherein the binding composition comprises a human or humanized antibody; a monoclonal antibody; a polyclonal antibody; an Fab fragment or an F(ab′)₂ fragment; a peptide mimetic of an antibody; a detectable label; or an anti-sense nucleic acid.

In another embodiment, the invention provides a method for the diagnosis of a proliferative condition comprising detecting or determining the expression or activity of at least one gene or polypeptide of Table 2, the above method wherein the gene is positive for Myc binding in a ChIP assay, the above method wherein the detecting or determining is by a binding composition comprising the antigen binding site from an antibody, a soluble receptor, or a nucleic acid, and the above method wherein the binding composition comprises a human or humanized antibody; a monoclonal antibody; a polyclonal antibody; an Fab fragment or an F(ab′)₂ fragment; a peptide mimetic of an antibody; a detectablc label; or a nucleic acid probe or nucleic acid primer.

Yet another aspect of the present invention is a method of treating a subject suffering from a proliferative disorder comprising administering to the subject an effective amount of an agonist or antagonist of at least one gene or polypeptide of Table 2, the above method wherein the gene is positive for Myc binding in a ChIP assay, and the above method wherein the proliferative disorder is oncogenic. The contemplated invention encompasses a method of treating a subject suffering from a proliferative disorder comprising administering to the subject an effective amount of an agonist or antagonist of at least one gene or polypeptide of Table 2, wherein the treating is by a binding composition, the above method wherein the binding composition comprises an antigen-binding site of an antibody, a soluble receptor, a nucleic acid, or a small molecule, and the above method wherein the binding composition comprises a human or humanized antibody; a monoclonal antibody; a polyclonal antibody; an Fab fragment or an F(ab′)₂ fragment; a peptide mimetic of an antibody; a detectable label; or an anti-sense nucleic acid.

### DETAILED DESCRIPTION

As used herein, including the appended claims, the singular forms of words such as “a,” “an,” and “the” include their corresponding plural references unless the context clearly dictates otherwise.

All references cited herein are incorporated herein by reference to the same extent as if each individual publication or patent application was specifically and individually indicated to be incorporated by reference.

I. Definitions.

“Activity” of a molecule refers, e.g., to binding of the molecule to a ligand or to a receptor, to catalytic activity, to the ability to stimulate, maintain, or inhibit gene expression, to antigenic activity, to the modulation of activities of other molecules, to modulation of ion transport, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like. Activity of a nucleic acid may refer to expression of a gene, e.g., rate of transcription from the gene, to rate of translation of an mRNA, or to concentration of the mRNA in a cell or tissue.

“Amino acid” refers to naturally occurring and synthetic amino acids, as well as to amino acid analogs and amino acid mimetics that function in a manner similar to the naturally occurring amino acids. Naturally occurring amino acids are those encoded by the genetic code, including selenomethionine, as well as those amino acids that are modified after incorporation into a polypeptide, e.g., hydroxyproline, γ-carboxyglutamate, O-phosphoserine, and cystine. Amino acid analogs refers to compounds that have the same basic chemical structure as a naturally occurring amino acid, i.e.; an alpha-carbon that is bound by a hydrogen, carboxyl group, amino group, and an R group. Amino acid analogs include, e.g., homoserine, norleucine, methionine sulfoxide, and methionine methyl sulfonium. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by their one-letter symbols.

“Angiogenesis” is the growth of new blood vessels in a tissue or organism.

“Antibody” refers to a polypeptide comprising a framework region from an immunoglobulin gene or fragments thereof that specifically recognizes and binds an antigen. The immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A “partially humanized” or “chimeric” antibody contains heavy and light chain variable regions of, e.g., murine origin, joined onto human heavy and light chain constant regions. A “humanized” or “fully humanized” antibody contains the amino acid sequences from the six complementarity-determining regions (CDRs) of the parent antibody, e.g., a mouse antibody, grafted to a human antibody framework. “Human” antibodies are antibodies containing amino acid sequences that are of 100% human origin, where the antibodies may be expressed, e.g., in a human, animal, bacterial, or viral host (Baca, et al. (1997) J. Biol. Chem. 272:10678-10684; Clark (2000) Immunol. Today 21:397-402).

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region.

“Fv” fragment comprises a dimer of one heavy chain and one light chain variable domain in tight association with each other. A single variable domain (or half of an Fv comprising only three CDRs specific for an antigen) has the ability to recognize and bind antigen, although at a lower affinity than the entire binding site.

“Monoclonal antibody” (mAb) refers to an antibody obtained from a population of substantially homogeneous antibodies, i.e., the individual antibody polypeptides comprising the population are identical except for possible naturally occurring mutations in the polypeptide chain that may be present in minor amounts. The term “monoclonal antibody” does not suggest any characteristic of the oligosaccharide component, or that there is homogeneity or heterogeneity with regard to oligosaccharide component. Monoclonal antibodies are highly specific, being directed against a single antigenic site or epitope. In contrast to conventional (polyclonal) antibody preparations which typically include different antibodies directed against different epitopes, each mAb is directed against a single determinant on the antigen. In addition to their specificity, the monoclonal antibodies are advantageous in that they-can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. “Monoclonal antibodies” also include clones of antigen-recognition and binding-site containing antibody fragments, such as those derived from phage antibody libraries.

“Diabodies” refers to a fragment comprising a heavy chain variable domain. (V_{H}) connected to a light chain variable domain (V_{L}) (Hollinger, et al. (1993) Proc. Natl. Acad. Sci. USA 90:6444-6448).

“Binding composition” refers to a molecule, small molecule, macromolecule, antibody, or a fragment or analogue thereof, or soluble receptor, capable of binding to a target. “Binding composition” also may refer to a complex of molecules, e.g., a non-covalent complex, to an ionized molecule, and to a covalently or non-covalently modified molecule, e.g., modified by phosphorylation, acylation, cross-linking, or cyclization, which is capable of binding to a target. “Binding composition” may also refer to a molecule in combination with a stabilizer, excipient, salt, buffer, solvent, or additive, capable of binding to a target. “Binding” may be defined as an association of the binding composition with a target where the association results in reduction in the normal Brownian motion of the binding composition, in cases where the binding composition can be dissolved or suspended in solution. “Modulating by a binding composition” can be effected by, e.g., treatment, administration, or contacting of a binding composition to a cell, host cell, cancer cell, tumor, tissue, organ, physiological fluid, research or clinical patient or animal. “Modulation” includes modulation of activity of, e.g., a gene, protein, polypeptide, or cellular function.

The phrase “specifically” or “selectively” binds, when referring to a ligand/receptor, antibody/antigen, or other binding pair, refers to a binding reaction which is determinative of the presence of the protein in a heterogeneous population of proteins and other biologics. Under designated conditions, a specified ligand binds to a particular, e.g., protein, receptor, or antigen, and binds to a lesser extent to other, e.g., protein, receptor, or antigen. The contemplated ligand or antibody of the invention binds to its target, e.g., a receptor or antigen, or a variant or miutein of the target, with an affinity that is generally two-fold greater, more generally four-fold greater, preferably 10-times greater, and still more preferably 20-times greater than the binding affinity to any other potential target. In a preferred embodiment the ligand or antibody will have an affinity which is greater than about 10⁹ liters/mol, as determined, e.g., by Scatchard analysis (Munsen, et al. (1980) Analyt. Biochem. 107:220-239).

“Cell line” refers to a population of cells capable of continuous or prolonged growth and division in vitro. Often, cell lines are clonal populations derived from a single progenitor cell. Spontaneous or induced changes can occur in the genome or can occur during storage or transfer of one or more cells present in the population of cells. Therefore, cells derived from the cell line referred to may not be precisely identical to the ancestral cells or cultures, and the cell line referred to includes such variants. The term “cell line” also includes immortalized cells (U.S. Pat. No. 6,090,611 issued to Covacci, et al.).

“Cell proliferation” is the rate of increase in cell number and is a function of the rate of cell division. Depending on the context, “cell proliferation” may indicate an overall increase in cell number, which is a function of cell division, cell death, or cell removal. Alternatively, cell proliferation may be used to indicate a quantity reflecting solely the rate of cell division. Proliferation may encompass phenomena such as the cell cycle, nutrient transport, growth, apoptosis; angiogenesis, and cell differentiation, where the phenomenon in question contributes to an increase in the rate of cell division or an increase in cell number.

Administration “in combination with” one or more therapeutic agents includes simultaneous or concurrent administration and consecutive administration, in any order.

“Chromatin” is the complex of genomic nucleic acids and proteins that can be found in the nucleus of the living cell, or in the cytoplasm of the cell when the nuclear membrane disappears, e.g., in mitosis or meiosis. The bound proteins include histones, modified histones, transcription factors, DNA polymerases, DNA repair proteins, and proteins controlling higher level structures of chromatin.

“Consensus E-boxes” and “non-consensus E-boxes” are defined (Blackwell, et al. (1993) Mol. Cell. Biol. 13:5216-5224; Grandori, et al. (1996) EMBO J. 15:4344-4357). The term E-box may refer to an E-box as it occurs in single stranded or in double stranded nucleic acids. Functional properties can provide guidance in defining E-boxes that vary somewhat from the consensus sequence, ie., E-boxes classed as non-consensus or non-canonical E-boxes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical nucleic acid sequences. Because of the degeneracy of the genetic code, a large number of functionally identical nucleic acids encode any given protein. As to amino acid sequences, one of skill will recognize that individual substitutions, deletions or additions to a nucleic acid, peptide, polypeptide, or protein sequence which alters, adds or deletes a conserved amino acid or a small percentage of amino acids in the encoded sequence is a “conservatively modified variant.” Conservative substitution tables providing functionally similar amino acids are well known in the art. An example of a conservative substitution is the exchange of an amino acid in one of the following groups for another amino acid of the same group (U.S. Pat. No. 5,767,063 issued to Lee, etal; Kyte and Doolittle (0.982) J. Mol. Biol. 157:105-132):

(1) Hydrophobic: Norleucine, Ile, Val, Leu, Phe, Cys, or Met;

(2) Neutral hydrophilic: Cys, Ser, Thr;

(3) Acidic: Asp, Glu;

(4) Basic: Asn, Gln, His, Lys, Arg;

(5) Residues that influence chain orientation: Gly, Pro;

(6) Aromatic: Trp, Tyr, Phe;

(7) Small amino acids: Gly, Ala, Ser.

“Detecting” generally relates to data that is or can be communicated or recorded as positive or negative, e.g., + or −, while “determining” generally relates to data that is or can be communicated or recorded as positive or negative, or in graded quantities, e.g., as −, +, ++, and +++, or in numerical quantities.

“Exogenous” refers to substances that are produced outside a cell, tissue, or organism, depending on the context. “Endogenous” refers to substances that are produced within a cell, tissue, or organism, depending on the context.

An “expression vector” is a nucleic acid construct, generated recombinantly or synthetically, with a series of specified nucleic acid elements that permit transcription of a particular nucleic acid. Typically, the expression vector includes a nucleic acid to be transcribed operably linked to a promoter.

“Gene expression” refers to transcription or translation, depending on the context. In transcription, mRNA is expressed from a gene. In translation, a polypeptide is expressed from mRNA.

An “immunoassay” is an assay that uses an antibody, antibody fragment, or antigen binding site derived from an antibody, to specifically bind an antigen. The immunoassay is characterized by the use of specific binding properties of a particular antibody to isolate, target, or quantify the antigen.

An “inhibitor” or “antagonist” refers, e.g., to a molecule, complex, or composition that reduces the activity of, e.g., a ligand, receptor, cofactor, nucleic acid, gene, cell, tissue or organ. An “activator” or “agonist” refers, e.g., to a molecule, complex, or composition that increases the activity of, e.g., a ligand, receptor, cofactor, nucleic acid, gene, cell, tissue or organ. “Modulator” refers to, e.g., a molecule, complex, or composition, that serves as an inhibitor or activator. The modulator cane act alone, or it may use a cofactor, e.g., a protein, metal ion, or small molecule. Inhibitors are compounds that decrease, block, prevent, delay activation, inactivate, desensitize, or down regulate, e.g., a gene, protein, or cell. An inhibitor may also be defined as a composition that reduces, blocks, or inactivates a constitutive activity. Activators are compounds that increase, activate, facilitate, enhance activation, sensitize, or up regulate, e.g., a gene, protein, or cell. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, even where there is no identified agonist.

To examine the extent of inhibition, samples or assay mixtures comprising, e.g., a given nucleic acid, polypeptide, cell, tissue, or organism, are treated with a potential activator or potential inhibitor and are compared to control samples without the inhibitor. Control samples, i.e., not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably 25% or less. Activation is achieved when the activity value relative to the control is about 110%, generally 120%, more generally 140%, more generally at least 160%, often 180%, more often 2-fold, most often 2.5-fold, usually 5-fold, more usually 10-fold, preferably 20-fold, more preferably 40-fold, and most preferably over 40-fold higher.

“Detectable inhibition” or “detectable decrease,” e.g., in expression of a gene or polypeptide of Tables 1 or 2, or of a predetermined activity, refers, e.g., to a comparison of expression or activity in the presence and absence of an agonist of a gene or polypeptide of Tables 1 or 2, or in the presence or absence of an antagonist of a gene or polypeptide of Tables 1 or 2. “Detectable” maybe a function of the context, e.g., of the reagents, instrumentation, or biological system. “Activity of a gene” may be defined as a rate, e.g., the rate of transcription, rate of translation, or as a concentration, e.g., concentration of the transcription or translation product in a cell, tissue, extract; or isolate.

Endpoints in activation or inhibition can be monitored as follows. Activation, inhibition, and response to treatment, of a cell, physiological fluid, tissue, organ, and animal or human subject, can be monitored by an endpoint. The endpoint may comprise a predetermined quantity or percentage of cell degranulation or secretion, e.g., of a cytokine, toxic oxygen, or a protease. Alternatively, the endpoint may comprise a predetermined quantity of ion flux, e.g., calcium flux, cell migration, cell adhesion, cell proliferation, potential for metastasis, cell differentiation, and change in phenotype, e.g., change, in expression of gene relating to inflammation, apoptosis, transformation, cell cycle, or metastasis, see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126). Generally, the endpoint of inhibition is 75% or less than the control, preferably the endpoint is 50% or less than the control, more preferably the endpoint is 25% or less than the control, and most preferably the endpoint is 10% or less than the control. Generally, the endpoint of activation is at least 150% control, preferably the endpoint is at least two times the control, more preferably the endpoint is at least four times the control, and most preferably the endpoint is at least 10 times the control.

A composition that is “labeled” is detectable, either directly or indirectly, by spectroscopic, photochemical, biochemical, immunochemical, isotopic, or chemical means. For example, useful labels include ³²P, ³³P, ³⁵S, ¹⁴C, ³H, ¹²⁵I, stable isotopes, fluorescent dyes and fluorettes (Rozinov and Nolan (1998) Chem. Biol 5:713-728; Molecular Probes, Inc. (2003) Catalogue, Molecular Probes, Eugene Oreg.), electron-dense reagents, enzymes and/or substrates, e.g., as used in enzyme-linked immunoassays as with those using alkaline phosphatase or horse radish peroxidase. The label or detectable moiety is typically bound, either covalently, through a linker or chemical bound, or through ionic, van der Waals or hydrogen bonds to the molecule to be detected. “Radiolabeled” refers to a compound to which a radioisotope has been attached through covalent or non-covalent means. A “fluorophore” is a compound or moiety that absorbs radiantenergy of one wavelength and emits radiant energy of a second, longer wavelength.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe can be detected by detecting the presence of the label bound to the probe. The probes are preferably directly labeled as with isotopes, chromophores, fluorophores, chromogens, or indirectly labeled such as with biotin to which a streptavidin complex or avidin complex can later bind.

“Ligand” refers to an entity that specifically binds to a polypeptide, to a complex comprising more than one polypeptide, or to a macromolecule such as a nucleic acid. A “ligand binding domain” or receptor is a region of, e.g., a polypeptide or nucleic acid, that is able to bind the ligand. A ligand can comprise, e.g., a soluble protein, membrane-associated protein, integral membrane-bound protein, oligosaccharide, lipid, or nucleic acid. Where a ligand binds to a receptor, the question of which molecule is the ligand and which molecule is the receptor can be determined on a case-by-case basis. Generally, where the binding event results in cell signaling, a molecule that is constitutively bound to the cell that responds to the signal may be considered to be part of the receptor, and not part of the ligand. A freely diffusable and water-soluble entity that is involved in ligand/receptor interactions is usually a ligand, not a receptor.

“Metastasis” is the process where a primary tumor mass spawns pioneer cells that invade adjacent tissues and travel to distant sites, where they found new colonies (Hanahan and Weinberg (2000) Cell 100157-70).

“Myc” refers to a family of genes and corresponding polypeptides. The Myc family includes c-Myc, N-Myc, L-Myc, S-Myc, and B-Myc. These proteins are most closely homologous at the MB1 and MB2 regions in the N-terminal region and at the basic helix-loop-helix leucine zipper (bHLHLZ) motif in the C-terminal region (Oster, et al. (2002) Adv. Cancer Res. 84:81-154; Grandori, et al. (2000) Annu. Rev. Cell Dev. Biol. 16:653-699). Myc also encompasses versions of Myc that are non, partially, and fully phosphorylated.

“Nucleic acid” refers to deoxyribonucleotides or ribonucleotides and polymers thereof, including single stranded and double stranded forms. The term encompasses nucleic acids containing nucleotide analogs or modified backbone residues or linkages. Examples of such analogs, e.g., phosphorothioates, phosphoramidates, and peptide-nucleic acids (PNAs).

A particular nucleic acid sequence also implicitly encompasses conservatively modified variants thereof, e.g., degenerate codon substitutions, and complementary sequences. “Nucleic acid” may be used to refer, e.g., to a gene, cDNA, mRNA, oligonucleotide, or polynucleotide. A particular nucleic acid sequence also implicitly encompasses, e.g., allelic variants, splice variants, and muteins.

“Nucleic acid probe” is a nticleic acid capable of binding to a target nucleic acid of complementary sequence, usually through complementary base pairing, e.g., through hydrogen bond formation. A probe may include natural, e.g., A, G, C, or T, or modified bases, e.g., 7-deazaguanosine, inosine, etc. The bases in a probe can be joined by a linkage other than a phosphodiester bond. Probes can be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

“Polymerase chain reaction” (PCR) refers, e.g., to a procedure or product where a specific region or segment of a nucleic acid is amplified, and where the segment is bracketed by pnriers used by DNA polymerase (Bernard and Wittwer (2002). Clin. Chem. 48: 1178-1185; Joyce (2002) Methods Mol. Biol. 193:83-92; Ong and Irvine (2002) Hematol. 7:59-67).

A “promoter” is a nucleic acid sequence that directs transcription of a nucleic acid. A promoter includes nucleic acid sequences near the start site of transcription, e.g., a TATA box, see, e.g., Butler and Kadonaga (2002) Genes Dev. 16:2583-2592; Georgel (2002) Biochem. Cell Biol. 80:295-300. A promoter also optionally includes distal enhancer or repressor elements, which can be located as much as several thousand base pairs on either side from the start site of transcription. A “constitutive” promoter is a promoter that is active under most environmental and developmental conditions, while an “inducible”, promoter is a promoter is active or activated under, e.g., specific environmental or developmental conditions. “Association of an E box with a promoter” means, e.g., that binding of Myc to/the E box results in a change in gene expression from that promoter, where the change may comprise, e.g., an increase or a decrease in the rate of gene expression.

“Protein” generally refers to the sequence of amino acids comprising a polypeptide chain. Protein may also refer to a three dimensional structure of the polypeptide. “Denatured protein” refers to a partially denatured polypeptide, having some residual three dimensional structure or, alternatively, to an essentially random three dimensional structure, i.e., totally denatured. The invention encompasses variants of proteins, and relevant methods, involving, e.g., glycosylation, phosphorylation, sulfation, disulfide bond formation, deamidation, isomerization, cleavage points in signal or leader sequence processing, covalent and non-covalently bound cofactors, oxidized variants, alternate folding, and the like. Disulfide links are described, e.g., see Woycechowsky and Raines (2000) Curr. Opin. Chem. Biol. 4:533-539; Creighton, et al. (1995) Trends Biotechnol. 13:18-23.

By “purified” and “isolated” is meant, when referring to a polypeptide, that the polypeptide is present in the substantial absence of the other biological macromolecules. The term “purified” as used herein, means typically about 70%, more typically 75%, at least 80%, ordinarily 85%, more ordinarily 90%, preferably 95%, and more preferably 98% by weight, or greater, of biological macromolecules present. The weights of water, buffers, salts, detergents, reductants, protease inhibitors, stabilizers, excipients, and other small molecules, especially those having a molecular weight of less than 1000, are generally not used in the determination of polypeptide purity (U.S. Pat. No. 6,090,611). Purity and homogeneity are typically determined using methods well known in the art (Scopes (1994) Protein Purification: Principles and Practice, Springer-Verlag, NY, N.Y., Cunico, Gooding, and Wehr (1998) Basic HPLC and CE of Biomolecules, Bay Biological Laboratory, Inc. Hercules, Calif.).

“Recombinant” when used with-reference, e.g., to a nucleic acid, cell, virus, plasmid, vector, or the like, indicates that these have been modified by the introduction of an exogenous, non-native nucleic acid or the alteration of a native nucleic acid, or have been derived from a recombinant nucleic acid, cell, virus, plasmid, or vector. Recombinant protein refers to a protein derived from a recombinant nucleic acid, virus, plasmid, vector, or the like.

“Small molecule” is defined as a molecule with a molecular weight that is less than 10 kD, typically less than 2 kD, and preferably less than 1 kD., Small molecules include, but are not limited to, inorganic molecules, organic molecules, organic molecules containing an inorganic component, molecules comprising a radioactive atom, synthetic molecules, peptide mimetics; and antibody mimetics. As a therapeutic, a small molecule may be more permeable to cells, less susceptible to degradation, and less apt to elicit an immune response than large molecules. Small molecule toxins are described, see, e.g., U.S. Pat. No. 6,326,482 issued to Stewart, et al.

“Soluble receptor” refers to receptors that are water-soluble and occur, e.g., in extracellular fluids, intracellular fluids, or weakly associated with a membrane. Soluble receptor also refers to receptors that are released from tight association with a membrane, e.g., by limited cleavage. Soluble receptor further refers to, receptors that are engineered to be water soluble, see, e.g., Monahan, et al. (1997) J. Immunol. 159:4024-4034; Moreland, et al. (1997) New Engl. J. Med. 337:141-141; Borish, et al. (1999) Am. J. Respir. Crit. Care Med. 160:1816-1823; Uchibayashi, et al. (1989) J. Immunol. 142:3901-3908.

“Treatment” refers to both therapeutic treatment and prophylactic or preventative measures.

II. General.

Myc is a transcription factor that binds to a specialized transcription regulation sequence known as an E-box, often resulting in increased gene expression. Deletion of E-boxes can result in decreased gene expression (Greasley, et al. (2000) Nucleic Acids Res. 28:446-453). Myc binds to a target gene by way of one or more E-boxes associated with that gene. However, no single target of Myc seems to account fully for Myc's biological effects, as several Myc targets appear to cooperate to maintain normal physiology, or to create cell transformation when Myc is overexpressed (Levens (2002) Proc. Natl. Acad. Sci. USA 99:5757-75759).

Myc plays a role in regulating cell proliferation, the cell cycle, cell growth, angiogenesis, apoptosis, and oncogenesis. Myc's activity can increase in tumors as a consequence of mutations, chromosomal rearrangemnents, increased expression, or gene amplification, e.g., see Nesbit, et al. (1999) Oncogene 18:3004-3016; Zeller, et al. (2001) J. Biol. Chem. 276:48285-48291; He, et al. (1998) Science 281:1509-1512; McMahon, et al. (1998) Cell 94:363-374; Erisman, et al. (1985) Mol. Cell. Biol. 5:1969-1976; Rochlitz, et al. (1996) Oncology 53:448-454. Elevated Myc activity in cancer cells may be a consequence of mutations in oncogenes other than Myc, e.g., APC or β-catenin (He, et al. (1998) supra). Increased Mye levels have been documented, e.g., in breast cancer and prostate cancer (Liao and Dickson (2000) Endocrine-Related Cancer 7:143-164; Jenkins, et al. (1997) Cancer Res. 57:524-531).

Myc regulates the cell cycle, growth, and apoptosis. Changes in cell cycle regulation can result in increased cell proliferation. When Myc regulates the cell cycle, it can act as a signaling agent that promotes entry of a cell into the cell cycle (Trumpp, et al. (2001) Nature 414:768-773; Holzel, et al. (2001) EMBO Reports 21:1125-1132; Bouchard, et al. (2001) Genes Devel. 15:2042-2047). Myc has been found to act in specific phases of the cell cycle, where certain cell cycle genes, e.g., cyclins and protein kinases, are directly or indirectly regulated by Myc (Oster, et al., supra). The invention provides methods for modulating the cell cycle.

Myc regulates growth, as it plays a role in regulating genes required for protein synthesis, e.g., genes for transcription factors and ribosomal proteins (Greasley, et al. (2000) supra; Zeller, et al. (2001) supra; Menssen and Hermeking (2002) Proc. Natl. Acad. Sci. USA 99:6274-6279). The invention contemplates methods for modulating growth.

Myc regulates apoptosis. Apoptosis can be impaired in cancer cells, as these cells are often able to avoid removal by cells of the immune system, survive in new locations in the body, or resist chemotherapy (Reed (2002) Apoptosis in The Cancer Handbook (Ed. by M. R. Alison) Nature Publishing Group, London, pp. 119-134). Myc regulates key apoptosis pathway proteins (Nesbit, et al. (1998) Blood 92:1003-1010; Oster, et al. (2002) supra). The contemplated invention provides methods for modulating apoptosis.

Techniques sensitive to the in vivo binding of Myc to candidate genes can identify Myc-target sites, as well as intracellular or extracellular factors that control Myc binding to these candidate genes. The invention identifies E-box containing Myc-target genes and polypeptides, and provides methods for modulating expression and activity of these genes and polypeptides for the treatment, of abnormal or pathologic cell proliferation, cell growth, metastasis, angiogenesis, and apoptosis (Pelangaris, et al. (2000) Curr. Opin. Genet. Dev. 10: 100-105). Provided are methods of modulating expression or activity of a nucleic acids or polypeptide of Tables, 1 and 2, as well as methods of diagnosing disorders or pathological conditions associated with a nucleic acid or polypeptide of Tables 1 and 2. These nucleic acids and polypeptides include, e.g., CLCN6, SLC4A2, CLNSIA, TAPK, and netrin-2 like protein.

CLCN6, SLC4A2, CLNSIA, and TAPK are ion transporters or ion channels (Tables 1 and 2). Ion transporters can modulate cell proliferation, apoptosis, and metastasis. Change in intracellular pH, eg., alkalinization, is a common feature of proliferating cells and tumor cells, where pH change results from changes in ion transporter activity. Ion transporter activity can serve as a checkpoint in the cell cycle. Chloride transporters can stimulate proliferation or cell invasiveness, as shown, e.g., by studies with chloride channel inhibitors. An additional role of ion transporters and cancer is function in extruding anti-cancer drugs, see, e.g., Elble and Pauli (2001) J. Biol. Chem. 276:40510-40,517; Szabo, et al. (1998) Proc. Natl. Acad. Sci. USA 95:6169-6174; Bustin, et al. (2001) DNA and Cell Biology 20:331-338; Soroceanu, et al. (1999) J. Neuroscience 19:5942-5954; Abdel-Ghany, et al. (2001) J. Biol. Chem. 276:25438-25446; Reshkin, et al. (2000) FASEB J. 2185-2197; Chien, et al. (2001) J. Cellular Biochem. 81:604-612; Wang, et al. (2002) J. Cellular Physiol. 193:1110-119; Ransom, et al. (2001) J. Neurosci. 21:7674-7683; Bustin; et al. (2001), DNA and Cell Biol. 20:331-338; Schlichter, et al. (1996) Glia 17:225-236; Blaisdell, et al. (1999) Am. J. Respir. Cell Mol. Biol. 20:842-847; Shen, et al. (2000) J. Physiol. (London) 529:385-394; Pappas and Ritchie (1998) Glia 22:113-120; Martinez-Zaguilan, et al. (1999) Biochem. Pharmacol. 57: 1037-1046.

CLCN6 (Tables. 1 and 2) is a chloride channel (Kornak, et al. (1999) Biochim. Biophys. Acia 1447:100-106). CLCN6 occurs near a position of the chromosome that is often deleted in cancer, e.g., ovarian, breast, colorectal cancer, and neuroblastoma (Gaughan, et al. (2000) Gene 257:279-289). The invention contemplates use of CLCN6 polypeptides and nucleic acids, antigenic fragments thereof, and binding compositions specific for CLCN6 polypeptides and nucleic acids, for the treatment and diagnosis of proliferative disorders, e.g., cancer.

SLC4A2 (Tables 1 and 2) is a chloride/bicarbonate anion exchanger (Lecanda, et al. (2000) Biochem. Biophys. Res. Commun. 276:117-124; Medina, et al. (2000) Biochem. Biophys. Res. Commun. 276:228-235; Medina, et. al. (1997) Genomics 39:74-85; Karet, et al. (1999) Am. J. Hum. Genet. 65: 1656-1665). Cl⁻/HCO₃⁻ exchangers modulate intracellular pH. The invention contemplates use of SLC4A2 polypeptides and nucleic acids, antigenic fragments thereof, and binding compositions specific for SLC4A2 polypeptides and nucleic acids, for the treatment and diagnosis of proliferative disorders, e.g., cancer.

CLNS1A (a.k.a. Icln; I_{cln}) (Tables 1 and 2) is a chloride transporter (Nagl, et al. (1996) Genomics 38:438-441; Scandella, et al. (2000) J. Biol. Chem. 275:15613-15620; Emma, et al. (2000) Am. J. Physiol. 274:C1545-C1551). CLNS1A resides on a region of the genome that is amplified in a subset of breast carcinomas prone to metastasis (Bekri, et al. (1997) Cytogenet. Cell Genet. (1997) 79:125-131). CLNS1A interacts with a protein (IBP72) that binds to a PAK-like kinase and appears to participate in cell cycling (Pu, et al. (2000) J. Biol. Chem. 275:12363-12366; Krapivinsky, et al. (1998) J. Biol. Chem. 273:10811-10814; Abe, et al. (1993) Biochim. Biophys. Acta 1173:353-356). The gene occurs in two locuses on the human genome, i.e., CLNS1A, which contains introns, and CLNS1B, which does not contain introns (Scandella, et al., supra). The E box of AF128461 occurs within an intron of human CLNS1A (GenBank NP_{—}001284; P54105) (Tables 1 and 2). The invention contemplates use of CLNS1A polypeptides and nucleic acids, antigenic fragments thereof, and binding compositions specific for CLNS1A polypeptides and nucleic acids, for the treatment and diagnosis of proliferative disorders, e.g., cancer.

Teratoma-associated tyrosine kinase (TAPK; gklp; ntkl) (Tables 1 and 2) resides in a region of the geno'me that contains breakpoints for chromosomal locations, where breakage occurs in various cancers. TAPK contains a protein kinase-like domain, but was found not to possess kinase activity. A mouse protein, p105, was found to be homologous to TAPK (van Asseldonk, et al. (2000) Genomics 66:35-42; Kato, et al. (2002) Genomics 79:760-767; Liu, et al. (2000) Biochim. Biophys. Acta 1517:148-152). The invention contemplates use of TAPK polypeptides and nucleic acids, antigenic fragments thereof, and binding compositions specific for TAPK polypeptides and nucleic acids, for the treatment and diagnosis of proliferative disorders, e.g., cancer.

Netrin-2 like protein (NTN2L) is a member of the netrin family of proteins, a family that includes netrin-1, nitrin-2, and netrin-3. The netrins, expressed by the nervous system, endocrine glands, muscle, and lungs, have been found to provide guidance to growing cells, e.g., axons, and to serve as a chemorepellent. NTN2L, a human netrin, is related to mouse netrin-3. Netrin-11 and netrin-3 bind to a number of receptors, e.g., DCC, neogenin, UNC5H1, UNC5H2, and LUNC5H3 (Schuldt (2003) Nature 422:125; Guthrie (1997) Current Biol. 7:R6-R9; Wang, et al. (1999) J. Neuroscience 19:4938-4947; Livesey (1999) Cell Mol. Life Sci. 56:62-68, Livesey and Hunt (1997) Mol. Cell. Neurosci. 8:417-429; Van Raay, et al. (1997) Genomics 41:279-282; Forcet, et al. (2002) Nature 417:443-447). DCC can mediate apoptosis or cell cycle arrest (Liu, et al. (2002) J. Biol. Chem. 277:26281-26285; Forcet, et al. (2001) Proc. Natl. Acad. Sci. USA 98:3416-3421). The invention contemplates use of NTN2L polypeptides and nucleic acids, antigenic fragments thereof, and binding compositions specific for NTN2L polypeptides and nucleic acids, for the treatment and diagnosis of proliferative disorders, e.g., cancer.

III. Myc Binding Assays.

Myc targets can be identified by methods sensitive to the binding of Myc to genomic target sequences, such as regulatory sequences containing an E-box. The chromatin immunoprecipitation (CHIP) method measures binding of Myc to target sequences. This method can involve pre-treating chromatin with formaldehyde to cross-link proteins to DNA, followed by limited fragmentation of chromatin, immunoprecipitation with anti-Myc antibody, with collection of immuno-precipitated genes or gene fragments, followed by their identification or quantitation, e.g., by the PCR method.

The nucleic acid sequences in non-precipitated and precipitated DNA can be identified by hybridization techniques or by PCR analysis, while the associated proteins can be identified by immunoblotting or amino acid sequencing (Menssen arid Hermeking (2002) supra; Boyd and Farnham (1999) Mol. Cell. Biol. 19:8389-8399; Boyd and Farnham (1997) Mol Cell. Biol. 17:2529-2537; Boyd, et al. (1998) Proc. Natl. Acad. Sci. USA 95:13887-13892; Frank, et al. (2001) Genes Devel. 15:2069-2082). Methods using electrophoretic mobility, microarrays, and transcription assays have also been used to identify Myc-targets (Oster, et al. (2002) supra; Schuhmacher, et al. (2001) Nucl. Acids Res. 29:397-406; Yu, et al. (2002) J. Biol. Chem. 277:13059-13666; Coller, et al. (2000) Proc. Natl. Acad. Sci. USA 97:3260-3265).

IV. Screening for Nucleic Acids, Polypeptides, and Binding Compositions.

Cells tissues, organs, or animals expressing a Myc-regulated, gene can be used for screening agents and compositions that modulate gene expression or activity of a polypeptide expressed from a gene of Tables 1 or 2. The cell or animal may comprise or express the natural Myc-regulated gene, or it can be engineered to comprise or express altered levels or muteins. Detection of endogenous and engineered genes in a cell, line, cell sample, or tissue sample generally involves detecting changes in levels of the relevant mRNA or polypeptide. Myc-regulated means, e.g., Myc-induced or Myc-suppressed.

Nucleic acids can be measured by methods dependent on hybridization, such as the TaqMan® technique, see, e.g., Heid, et al. (1996) Genome Res. 6:989-994; Liu, et al. (2002) Analyt. Biochem. 300:40-45; Huang, et al. (2000) Cancer Res. 60:6868-6874; Wittwer, et al. (1997) Biotechniques 22:130-138; Schmittgen, et al. (2000) Analyt. Biochem. 285:194-204; Sims, et al. (2000) Analyt. Biochem. 281:230-232.

Microarrays can be used for screening, see, e.g., Ausubel, et al. (2001) Curr. Protocols Mol. Biol., Vol. 4, John Wiley and Sons, New York, N.Y., pp. 22.0.1-22.3.26; (Huang, et al. (2000) Cancer Res. 60:6868-6874; Ausubel, et al. (2001) Curr. Protocols Mol. Biol., Vol. 4, John Wiley and Sons, New York, N.Y., pp. 25.0.1-25B.2.20; Ausubel, et al. (2001) Curr. Protocols Mol. Biol., Vol. 3, John Wiley and Sons, New York, N.Y., pp. 14.0.1-14.14.8; Gray, et al. (1998) Science 281:533-538; U.S. Pat. No. 6,028,186 issued to Tasset, et al.

Methods for screening and assessing properties of enzymes, e.g., protein kinases are available, see, e.g., Al-Obeidi and Lam (2000) Oncogene 19:5690-5701; Ohmi, et al. (2000) J. Biomol. Screen. 5:463-470; Chapman and Wong (2002) Bioorganic Medicinal Chem. 10:551-555; Stratowa, et al. (1999) Anti-Cancer Drug Design 14:393-402.

Cells can be screened and purified, e.g., by fluorescent activated cell sorting (FACS), see, e.g., Melamed, et al. (1990) Flow Cytometry and Sorting, Wiley-Liss, Inc., New York, N.Y.; Shapiro (1988) Practical Flow Cytometry, Liss, New York, N.Y., and Robinson, et al. (1993) Handbook of Flow Cytometry Methods, Wiley-Liss, New York, N.Y.

V. Protein Purification

It is contemplated to purify the polypeptide diagnostics or therapeutics used in the methods of the invention, e.g., antigens, antibodies, and antibody fragments, by methods that are established in the art. Purification can be accomplished by, e.g., immunoprecipitation, ion, exchange chromatography, epitope tags, affinity chromatography, and high pressure liquid chromatography, with optional use of detergents, emulsifiers, and stabilizing agents, see, e.g., Dennison and Lovrien (1997) Protein Expression Purif 11:149-161; Murby, et al. (1996) Protein Expression Purif. 7:129-136; Ausubel, et al. (2001) Curr. Protocols Mol. Biol, Vol. 3, John Wiley and Sons, New York, N.Y., pp. 17.0.1-,17.23.8; Rajan, et al. (1998) Protein Expression Purif. 13:67-72; Amersham-Pharmacia (2001) Catalogue, Amersharm-Pharmacia Biotech, Inc., pp. 543-567, 605-654; Gooding and Regnier (2002) HPLC of Biological Molecules, 2nd ed., Marcel Dekker, NY.

VI. Small Molecule Therapeutics.

The invention encompasses use of small molecule diagnostics and therapeutics for, e.g., modulating expression and activity of Myc-binding genes or the respective gene products (Tables 1 or 2). Natural products and synthetic compounds are generally known as “small molecules” when of significantly lesser molecular weight than a typical polypeptide, i.e., significantly lower than 50 kDa. Methods for preparing and using small molecules are described, see, e.g., Al-Obeidi and Lam (2000) Oncogene 19:5690-5701; Bishop, et al. (2001) Trends Cell Biol 11:167-172; Traxler, et al. (2001) Med. Res. Revs. 21:499-512; Gray, et al. (1998) Science 281:533-538; Stratowa, et al. (1999) Anti-Cancer Drug Design 14:393-402; Rosen (2001) Cancer J., 7 Suppl. 3:5120-128; Sawyers (2002) Curr. Op. Genetics Devel. 12:111-115; Rosen (2001) Cancer J. 7 Suppl. 3:S120-128; Ripka and Rich (1998) Curr. Opinion Chemical Biol. 2:441-452; Hruby, et al. (1997) Curr. Opinion Chemical Biol. 1:114-119; al-Obeidi, et al. (1998) Mol. Biotechnol. 9:205-223; Hruby and Balse (2000) Curr. Med. Chem. 7:945-970, Martin-Moe, et al. (1995) Pept. Res. 8:70-76; Guichard, et al. (1994) Proc. Natl. Acad. Sci. USA 91:9765-9769; Sloan (1992) Prodrugs, Marcel Dekker, New York, N.Y.; Melton and Knox (1999) Enzyme-Prodrug Strategies for Cancer Therapy, Plenum Publ. Corp., New York, N.Y.; U.S. Pat. No. 6,326,482 issued to Stewart, et al. The invention also contemplates the use of pro-drugs, see, e.g., Iyengar, et al. (2002) Cancer Res. 61:3045:3052; Nishino, et al. (1999) J. Biol. Chem. 274:32580-32587; Pawlik, et al. (2000) Mol. Ther. 1:457-463.

VII. Antibodies.

Antibodies can be raised to a polypeptide gene product, or an antigenic fragment, of a polypeptide of Table 1 or 2, to a chromosomal protein associated with a Myc-binding site or gene of Tables 1 or 2, to biologically or catalytically active or inactive polypeptides, and to native or denatured polypeptides. Anti-idiotypic antibodies are also contemplated.

Antigenic sequences of the polypeptides corresponding to the genes of Table 2 were determined by a Parker plot using Vector NTI® Suite, Informax, Inc., Bethesda, Md. (Parker, et al. (1986) Biochemistry 18:5425-5431).

CLCN6 has regions of increased antigenicity at, e.g., amino acids 15-29, 33-42, 46-56, 67-79, 115-124, 232-240, 320-326, 397-412, and 667-694, of AF009247 (Tables 1 and 2).

CLNS1A has regions of increased antigenicity at, e.g., amino acids at 18-25, 95-108, 137-163, 212-225, of NP_{—}001284 or P54105). NP_{—}001284 or P54105 is the polypeptide of the gene containing the intron of AF148461 (Tables 1 and 2).

SLC4A2 has regions of increased antigenicity at, e.g., amino acids 91-138, 180-204, 290-323, and 561-578, of U76667 (Tables 1 and 2).

TAPK has regions of increased antigenicity at, e.g., amino acids 18-28, 114-124, 134-140, 318-326, and 398-413, of AF255613 (Tables 1 and 2).

Netrin-2-like protein has regions of increased antigenicity at, e.g., amino acids 20-41, 57-78, 225-238, 275-291, and 381-401, of U86758 (Tables and 2).

Antibodies and binding compositions derived from an antigen-binding site of an antibody are provided. These include human antibodies, humanized antibodies, monoclonal antibodies, polyclonal antibodies, and binding fragments, such as Fab, F(ab)₂, and Fv fragments, and engineered versions thereof. The antibody or binding composition can be agonistic or antagonistic. Antibodies that simultaneously bind to a ligand and receptor are contemplated. Monoclonal antibodies will usually bind with a K_{D} of 1 mM or less, more usually 300 μM or less, typically 100 μM or less, more typically 30 μM or less, preferably at 10 μM or less, and most preferably at 3 μM or less.

Antibodies can be prepared, see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang, et al. (1999) J. Biol. Chem. 274:27371-27378.

A humanized antibody contains the amino acid sequences from-six complementarity determining regions (CDRs) of the parent mouse antibody, which are grafted on a human antibody framework. An alternative to humanization is to use human antibody libraries displayed on phage or human antibody libraries contained in transgenic animals or cells, see, e.g., Vaughan, et al. (1996) Nat. Biotechnol. 14:309-314; Barbas (1995) Nature Med. 1:837-839; de Haard, et al. (1999) J. Biol. Chem. 274:18218-18230; McCafferty et al. (1990) Nature 348:552-554; Clackson et al. (1991) Nature 352:624-628; Marks et al. (1991) J. Mol. Biol. 222:581-597; Mendez, et al. (1997) Nature Genet. 15:146-156; Hoogenboom and Chames (2000) Immunol. Today 21:371-377; Barbas, et al. (2001) Phage Display: A Laboratory Manual, Cold Spring Harbor Laboratory Press, Cold Spring Harbor, N.Y.; Kay, et al. (1996) Phage Display of Peptides and Proteins: A Laboratory Manual, Academic Press, San Diego, Calif.; de Bruin, et al. (1999) Nat. Biotechnol. 17:397-399.

Single chain antibodies, single domain antibodies, and bispecific antibodies are described, see, e.g., Malecki, et al. (2002) Proc. Natl. Acad. Sci. USA 99:213-218; Conrath, et al. (2001) J. Biol. Chem. 276:7346-7350; Desmyter, et al. (2001) J. Biol. Chem. 276:26285-26290, Kostelney, et al. (1992) J. Immunol. 148:1547-1553; U.S. Pat. Nos. 5,932,448; 5,532,210; 6,129,914; 6,133,426; 4,946,778.

Antigen fragments can be joined to other materials, such as fused or covalently joined polypeptides, to be used as immunogens. An antigen and its fragments can be fused or covalently linked to a variety of immunogens, such as keyhole limpet hemocyanin, bovine serum albumin, or ovalbumin (Coligan, et al. (1994) Current Protocols in Immunol., Vol 2, 9.3-9.4, John Wiley and Sons, New York, N.Y.). Peptides of suitable antigenicity can be selected from the polypeptide target, using an algorithm, such as those of Parker, et al. (1986) Biochemistry 25:5425-5432; Jameson and Wolf (1988) Cabios 4:181-186; or Hopp and Woods (1983) Mol. Immunol. 20:483-489.

Purification of antigen is not necessary for the generation of antibodies. Immunization can be performed by DNA vector immunization or by immunization with cells bearing the antigen of interest. Immunization with cells may prove superior for antibody generation than immunization with purified antigen, see, e.g., Wang, et al. (1997) Virology 228:278-284; Meyaard, et al. (1997) Immunity 7:283-290; Wright, et al. (2000) Immunity 13:233-242; Preston, et al. (1997) Eur. J. Immunol. 27:1911-1918; Kaithamana, et al. (1999) J. Immunol. 163:5157-5164.

Antibody to antigen binding properties can be measured, e.g., by surface plasmon resonance or enzyme linked immunosorbent assay (ELISA) (Karlsson, et al. (1991) J. Immunol. Methods 145:229-240; Neri, et al. (1997) Nat. Biotechnol. 15:1271-1275; Jonsson, et al. (1991) Biotechniques 11:620-627; Friguet, et al. (1985) J. Immunol. Methods 77:305-319; Hubble (1997) Immunol. Today 18:305-306).

Antibodies to polypeptides, or to antigenic fragments thereof, expressed from the genes of Tables 1 or 2 but possessing substitutions that do not substantially affect the functional aspects of the nucleic acid or amino acid sequence, are within the definition of the contemplated invention. Variants with truncations, deletions, additions, and substitutions of regions which do not substantially change the biological functions of these nucleic acids and polypeptides are also within the definition of the contemplated invention.

VIII. Therapeutic Compositions.

The invention provides methods to treat and diagnose various proliferative conditions, e.g., cancer, tumors, metastasis, and angiogenesis.

Formulations of antibodies, binding composition, polypeptides, antibody mimetics, or small molecule therapeutics, e.g., antisense nucleic acids, are prepared for storage by mixing with physiologically acceptable carriers, excipients, or stabilizers in the form of, e.g., lyophilized powders, slurries, aqueous solutions, see, e.g., Hardman, et al. (2001) Goodman and Gilman 's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.; D'Alessandro (1993) Cancer Therapy: Differentiation, Immunomodulation and Angiogenesis, Springer-Verlag, New York, N.Y.; U.S. Pat. Nos. 6,096,728; 6,342,220; and 5,440,021.

Therapeutic compositions comprising an antibody or small molecule can be administered, e.g., by systemic, intraperitoneal, intramuscular, dermal, subcutaneous, oral, nasal, pulmonary, suppository, and intratumor routes. Sustained-release preparations, liposomes, aerosols, or viral vectors can supply the therapeutic composition by the contemplated method, see, e.g., Sidman et al. (1983) Biopolymers, 22:547-556; Langer et al. (1981) J. Biomed. Mater. Res. 15:167-277, Langer (1982) Chem. Tech. 12:98-105; Lasic and Papahadjopoulos (eds.) (1998) Medical Applications of Liposomes, Elsevier Health Sciences, Phila., Pa.; Janoff (ed.) (1999) Liposomes: Rational Design, Marcel Dekker, Inc., NY, N.Y.; Knowles, et al. (1995) New Engl. J. Med. 333:823-831; U.S. Pat. Nos. 6,387,404 and 6,375,972.

An “effective amount” of antibody or other therapeutic, or diagnostic, to be employed will depend, i.e., upon the objectives, the route of administration, the type of antibody employed, and the condition of the patient or subject. Accordingly, it will be necessary for the therapist to titer the dosage and modify the route of administration as required to obtain the optimal therapeutic effect. Typically, the clinician will administer the antibody until a dosage is reached that achieves the desired effect. The progress of this therapy is easily monitored by conventional assays. An effective amount of therapeutic will decrease the symptoms typically by at least about 10%; usually by at least 20%; preferably at least about 30%; more preferably at least about 50%; and most preferably at least about 90%. Guidance in therapeutic and diagnostic methodology is available, see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK.,

As a general proposition, the initial, pharmaceutically effective amount of the antibody administered parenterally will be in the range of about 0.1 μg/kg to 10 mg/kg of the patient's body weight per day, ordinarily 0.1 μg/kg/day to 1.0 mg/kg/day; preferably 0.1 μg/kg/day to 0.1 mg/kg/day, more preferably 0.1 μg/kg/day to 0.01 mg/kg/day, and most preferably 0.1 μg/kg/day, or less. The desired dosage can be delivered by a single bolus administration, by multiple bolus administrations, or by continuous infusion administration of antibody, depending on the pattern of pharmacokinetics that the practitioner wishes to achieve. These suggested amounts of antibody are subject to a fair amount of therapeutic discretion. The key factor in selecting an appropriate dose and scheduling is the result obtained.

In the treatment and prevention of an inflammatory disorder the therapeutic composition will be formulated, dosed, and administered in a fashion consistent with good medical practice. The “therapeutically effective amount” of antibody or binding composition to be administered will be the minimum amount necessary to prevent, ameliorate, or treat the inflammatory or proliferative disorder while minimizing possible toxic effects to the host or patient.

IX. Kits.

The invention provides methods of using the Myc-binding genes of Tables 1 or 2, expressed nucleic acids, expressed polypeptides, and binding compositions thereto, in a diagnostic kit. Also encompassed is use of antigenic fragments, muteins, metabolites, and chemical and metabolic breakdown products of the polypeptides of Tables 1 or 2. Typically, the kit will have a compartment containing a polypeptide of Tables 1 or 2, or an antigenic fragment thereof, a binding composition, or a nucleic acid, e.g., a nucleic acid probe or primer.

The kit can comprise, e.g., a reagent and a compartment, a reagent and instructions for use, or a reagent with both a compartment and instructions for use. The reagent can comprise a polypeptide of Tables 1 or 2, or an antigenic fragment thereof, a binding composition, or a gene or nucleic acid of Tables 1 or 2. A kit for determining the binding of a test compound or test binding composition to a target can comprise a control compound, a labeled compound, and a method for separating free labeled compound from bound labeled compound. Conjugated antibodies are useful for diagnostic or kit purposes, and include, e.g., antibodies coupled to dyes, isotopes, enzymes, and metals, see, e.g., Le Doussal, et al. (1991) J. Immunol. 146:169-175; Gibellini, et al. (1998) J. Immunol. 160:3891-3898; Using and Bishop (1999) J. Immunol. 162:2804-2811; Everts, et al. (2002) J. Immunol 168:883-889. Diagnostic assays can be used with biological matrices such as live cells, cell extracts or cell lysates, fixed cells, cell cultures, bodily fluids, or forensic samples. Various assay formats are available, e.g., radioimmunoassays (RIA), ELISA, and lab on a chip (U.S. Pat. Nos. 6,176,962 and 6,517,234). Numerous methods are available for separating bound ligand from free ligand, or bound test compound from free test compound, e.g., use of ligands or test compound immobilized by adhesion to plastic, and couplings involving a complex of antigen and antibody, biotin and avidin, and biotin and streptavidin.

X. Uses.

The present invention provides methods and reagents that will find use in therapeutic and diagnostic applications, e.g., for the treatment and diagnosis of cancer and other proliferative conditions. A reagent sensitive to a single Myc-binding gene or gene product, or to a group of Myc-binding genes or gene products of Tables 1 or 2, is expected to be useful as a: probe in antibody-based assays, FACS assays, histological assays, nucleic acid hybridization-based assays, PCR-based assays, and the like.

The invention provides a binding composition specific for at least one ion transporter, e.g., CLCN6, CLN1 SA, and SLC4A2, specific for at least one protein kinase, e.g., TAPK; or specific for at least one agent that guides cell growth, guides the direction of cell growth, or modulates apoptosis, e.g., NTN2L (Tables 1 and 2).

The broad scope of this invention is best understood with reference to the following examples, which are not intended to limit the inventions to the specific embodiments.

### EXAMPLES

I. General Methods.

Some of the standard methods are described or referenced, see e.g., Maniatis, et al. (1982) Molecular Cloning, A Laboratory Manual, Cold Spring Harbor Laboratory, Cold Spring Harbor Press; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.; Innis, et al. (eds.) (1990) PCR Protocols: A Guide to Methods and Applications, Academic Press, N.Y. Standard methods are also found in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification such as immunprecipitation, column chromatography, electrophoresis, isoelectric focusing, centrifugation, and crystallization, are described (Coligan, et al. (2000) Current Protocols in Protein Science, Vol. 1, John Wiley and Sons, Inc., New York). Chemical analysis, chemical modification, post-translational modification, and glycosylation of proteins is described (Coligan, et al. (2000) Current Protocols in Protein Science, Vol. 2, John Wiley and Sons, Inc., New York). The production, purification, and fragmentation of polyclonal and monoclonal antibodies is described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.); Harlow and Lane (1988) supra).

Cell culture techniques are described in Doyle, et al. (eds.) (1994) Cell and Tissue Culture: Laboratory Procedures, John Wiley and Sons, NY. FACS analysis is described in Melamed, et al. (1990) Flow Cytometry and Sorting, Wiley-Liss, Inc., New York, N.Y.; Shapiro (1988) Practical Flow Cytometry, Liss, New York, N.Y.; and Robinson, et al. (1993) Handbook of Flow Cytometry Methods, Wiley-Liss, New York, N.Y.

Methods for the diagnosis and treatment of cancer and other proliferative disorders, angiogenesis, and apoptosis, are described (Warrington, et al. (2002) Microarrays and Cancer Research, Eaton Pub. Co. Natick, M A; Fletcher (2000) Diagnostic Histopathology of Tumors, Churchill Livingstone, St. Louis, Mo.; Chabner (2001) Cancer Chemotherapy and Biotherapy, 3^{rd} ed., Lippincott Williams and Wilkins, Phila., Pa.; Casciato and Lowitz (2000) Manual of Clinical Oncology, 4^{th} ed., Lippincott, Williams, and Wilkins, Phila., Pa.; Rubanyi (2000) Angiogenesis in Health and Disease, Marcel Dekker, New York, N.Y.; Cotter (1997) Techniques in Apoptosis, Univ. of Calif. Press, Berkeley, Calif.; Leblanc (2002) Apoptosis: Techniques and Protocols, Humana Press, Totawa, N.J.; Hughes and Mehmet (2002) Cell Proliferation and Apoptosis, Springer Verlag, New York, N.Y.

II. Gene Fragments Producing a Positive Signal in the ChIP Assay.

2224 E-boxes were screened by the ChIP assay. The E-boxes and associated human genes that screened positive are shown (Table 1). The structure of the E-box associated with each gene is shown in the Abstract of Mol, et al. (1995) Mol. Cell. Biol. 15:6999-7009. U937 cells, HL60 cells, P496-3 cells (−Tet), P496-3 cells (+Tet), T98G cells, and WS1 cells served as the source of chromatin in the ChIP assays. A separate group of E-boxes, randomly chosen from chromosome 21, was subjected to the ChIP test, using anti-Myc as the test antibody. The results from these randomly chosen samples served as a control.

Following immunoprecipitation and quantitation of the immunoprecipitated gene by the PCR technique, the following calculations were made: (1) Comparison of PCR signal from chromatin collected with anti-Myc antibody (experimental) with PCR signal from unfractionated chromatin, expressed as a percentage; (2) Comparison of PCR signal from chromatin subjected to a control immunoprecipitation (control) with PCR signal from unfractionated chromatin, expressed as a percentage; (3) Comparison of (1) with (2) to provide an apparent value for “fold enrichment.”

Two criteria must be satisfied for a gene/E box to be considered to screen positive in the ChIP assay. First, the PCR signal from the +anti-Myc antibody ChIP test (see (1) above) must be 0.1% or greater than that of the PCR signal from the unfractionated, sonicated chromatin, for a given gene target. Second, comparison between (1) and (2) (see above) must be 10-fold or greater. In other words, a site was classed as positive if it was enriched by at least 0.1% in Myc immunoprecipitates and was enriched by at least 10-fold over its value in control precipitates. A gene satisfying both criteria was considered to give a positive result by the ChIP assay. Positively screening genes/E boxes are listed in Table 1.

Alternate definitions for a positive-screening ChIP result may be devised, e.g, those that are more or less stringent than the above definition.

All of the genes of Table 1 show a “fold-enrichment value” of 10-fold or greater and gave a PCR signal from the +anti-Myc antibody ChIP test (once recovered in the immunoprecipitate) that was 0.1% or greater than that of the PCR signal from the unfractionated, sonicated chromatin (Table 1). Negative controls in the ChIP assays include genes containing no E-boxes, e.g., PCNA, acetylcholine receptor, and topoisomerase II, and genes containing an E-box but where Myc did not bind, e.g., glucokinase; glycine methyltransferase; socs-2 (Frank, et al. (2001) supra).

TABLE 1

| Accession | | Position of |
|---|---|---|
| Number | Gene Definition | E-box |
| AB004270_p1 | hMCM7 gene promoter region. | −74 |
| AB004270_p1 | hMCM7 gene promoter region. | −74 |
| AB009271_p1 | BCNT, partial cds. | 915 |
| AB009589_i1 | Osteomodulin | 5231 |
| AB009666_p1 | Klotho gene, exon 1. | 943 |
| AB012942_i1 | AOC2, retina-specific amine oxidase, exon 1 | 3353 |
| AB012943_p1 | AOC2 gene for retina-specific amine oxidase, exons 2a, 2b, 3 | 1038 |
| | and 4 and complete cds | |
| AB013139_p1 | NBS1, complete cds. | 1001 |
| AB014460_p1 | TSC2 and NTHL1 head-to-head genes | −1368 |
| AB014460_p1 | TSC2 and NTHL1 head-to-head genes | −9 |
| AB014460_p1 | TSC2 and NTHL1 head-to-head genes | 289 |
| AB015961_i1 | IL-18, intron 1 and exon 2. | −3195 |
| AB015961_i1 | IL-18, intron 1 and exon 2. | −2819 |
| AB016194_p1 | Elk1 oncogene, complete cds. | −4 |
| AB016194_p1 | Elk1 oncogene, complete cds. | 1599 |
| AB016243_p1 | Regulatory factor 2 of sodium/hydrogen exchanger isoform A3, | 657 |
| | complete cds | |
| AB016243_p1 | Regulatory factor 2 of sodium/hydrogen exchanger isoform A3, | 820 |
| | complete cds | |
| AB017018_p1 | JKTBP2, JKTBP1, complete cds. | −1147 |
| AB017018_p1 | JKTBP2, JKTBP1, complete cds. | −939 |
| AB017710_p1 | U50HG genes for U50′ snoRNA and U50 snoRNA, complete | −562 |
| | sequence. | |
| AB017710_p1 | U50HG genes for U50′ snoRNA and U50 snoRNA, complete | 161 |
| | sequence | |
| AB019198_i1 | Caspase-9, exon 2. | −98 |
| AB019581 | wee1 | 166 |
| AB019581 | wee1 | −38 |
| AB019581 | wee1 | −832 |
| AB020236 | RPL27a | 424 |
| AB028893_p2 | RPL13A, U32, U33, U34, U35, RPS11, U35 genes for | 490 |
| | ribosomal protein L13a and S11, U32, U33, U34, U35, and U35 | |
| | snoRNA complete cds and sequence | |
| AB028893_p2 | RPL13A, U32, U33, U34, U35, RPS11, U35 genes for | −220 |
| | ribosomal protein L13a and S11, U32, U33, U34, U35, and U35 | |
| | snoRNA complete cds and sequence | |
| AB030817_p1 | Hper1 gene for period1, complete cds. | −1360 |
| AB030817_p1 | Hper1 gene for period1, complete cds. | −629 |
| AB032481_p1 | HOXD13 gene for homeobox transcription factor, complete cds | 691 |
| AB038980, | caspase 8 | 202 |
| NT_005229 | | |
| AB038980, | caspase 8 | 558 |
| NT_005229 | | |
| AF210257, | caspase 8 | 24829 |
| NT_005229 | | |
| AF210257, | caspase 8 | 31045 |
| NT_005229 | | |
| AF210257, | caspase 8 | 32634 |
| NT_005229 | | |
| AB046408 | RPL22 | 74 |
| AC007114 | AKAP1 | −1994 |
| AC007114 | AKAP1 | −15 |
| AC007114 | AKAP1 | 900 |
| AC008532 | PUMA/JFY1/BBC3 | 773 |
| AC008532 | PUMA/JFY1/BBC3 | 1628 |
| AF332559 | PUMA/JFY1/BBC3 | −379 |
| AF411827 | PUMA/JFY1/BBC3 | −1705 |
| AF001689 | RPL23a | 2055 |
| AF001976 | Type I sigma receptor | 396 |
| AF005891 | Na K-ATPase beta-3 subunit (atp1b3), exon 2. | intron 1 |
| | | (3′end) |
| AF009247_p1 | Putative chloride channel gene (CLCN6), partial promoter and | −91 |
| | exon 1 | |
| AF009247_p1 | Putative chloride channel gene (CLCN6), partial promoter and | −68 |
| | exon 1 | |
| AF009247_p1 | Putative chloride channel gene (CLCN6), partial promoter and | 244 |
| | exon 1 | |
| AF010238_p1 | von Hippe1-Lindau tumor suppressor (VHL) gene | 911 |
| AF010317_p1 | Pig3 (PIG3) gene, partial cds. | 1269 |
| AF015947 | cad gene, promoter region. | −2025 |
| AF015947_p1 | cad gene, promoter region. | −454 |
| AF015947_p1 | cad gene, promoter region. | −483 |
| AF018631 | Biotinidase (BTD), exons 2-4 | −364 |
| AF025878_p1 | Inosine monophosphatase 2 (IMPA2) gene, promoter sequence | −690 |
| AF026855 | Mitochondrial short-chain L-3-hydroxyacyl-CoA dehydrogenase | intron 1 |
| | (HADHSC) | |
| AF029081 | 14-3-3 sigma protein promoter and gene | −8169 |
| AF029081 | 14-3-3 sigma protein promoter and gene | −7215 |
| AF033033_p1 | TIRC7 protein (TCIRG1) gene, complete cds. | 603 |
| AF035427_p1 | Bone morphogenetic protein 4 (BMP4) gene, promoter 1 and | −196 |
| | exon 1 | |
| AF035753_p1 | Endoglin (END) gene, promoter region, exon 1 and partial cds | −1001 |
| AF035753_p1 | Endoglin (END) gene, promoter region, exon 1 and partial cds | 338 |
| AF039954_p1 | CC chemokine LCC-1 precursor, gene, complete cds. | −147 |
| AF042836 | Cystathionine beta-synthase (CBS) gene, major and minor | −1588 |
| | alternative splice products, complete cds | |
| AF042836 | Cystathionine beta-synthase (CBS) gene, major and minor | −573 |
| | alternative splice products, complete cds | |
| AF042836 | Cystathionine beta-synthase (CBS) gene, major and minor | 737 |
| | alternative splice products, complete cds | |
| AF042836 | Cystathionine beta-synthase (CBS) gene, major and minor | 1434 |
| | alternative splice products, complete cds | |
| AF042848_p1 | EMMPRIN gene, promoter and exon 1. | −228 |
| AF046925_p1 | Hepatocyte growth factor receptor (c-met) gene, promoter | −385 |
| | sequence | |
| AF049198_i1 | Sodium iodide symporter gene, intron 1, 5′ partial | intron 1 |
| AF050068_p1 | Growth arrest specific 11 GAS11) gene, exon 1. | −208 |
| AF050115_p1 | Hypoxia-inducible factor 1 alpha subunit (HIF1A) gene, exon 1 | −276 |
| AF050115_p1 | Hypoxia-inducible factor 1 alpha subunit (HIF1A) gene, exon 1 | −276 |
| AF055992_p1 | Duffy antigen/chemokine receptor (FY) gene, FY*X allele, | −568 |
| | complete cds | |
| AF059650_p1 | Histone deacetylase 3 (HDAC3) gene, complete cds. | −1525 |
| AF060494_p1 | Ubiquitin binding protein p62 gene, promoter and partial cds | −1344 |
| AF065396_p1 | Retinoic X receptor B gene, complete cds. | 796 |
| AF067130_p1 | Protein phosphatase-1 regulatory subunit 7 (PPP1R7) gene, exon 1 | 469 |
| AF067572_p1 | Signal transducer and activator of transcription 6 (STAT6) gene, | −1754 |
| | exons 1 through 12 | |
| AF067572_p1 | Signal transducer and activator of transcription 6 (STAT6) gene, | −823 |
| | exons 1 through 12 | |
| AF067572_p1 | Signal transducer and activator of transcription 6 (STAT6) gene, | −148 |
| | exons 1 through 12 | |
| AF067844_p1 | Chromosome 10 clone PTEN, complete sequence. | −1374 |
| AF067844_p1 | Chromosome 10 clone PTEN, complete sequence. | −1148 |
| AF069984_p1 | Nitrilase homolog 1 (NIT1) gene, alternatively spliced, product, | −621 |
| | complete cds | |
| AF069984_p1 | Nitrilase homolog 1 (NIT1) gene, alternatively spliced product, | 1459 |
| | complete cds | |
| AF071540_p1 | Erythrocyte membrane protein 4.2 gene, promoter sequence | −316 |
| AF071541 | Erythrocyte membrane protein 4.2 gene, intron 1 | intron 1 |
| AF072562_p1 | Zinc finger protein 74 (ZNF74) gene, exons 2a and 2b. | −40 |
| AF074333_p1 | Glycogen synthase kinase 3 beta gene, promoter region and | −323 |
| | partial cds | |
| AF076613 | Promyelocytic leukemia zinc finger (PLZF) | 1299 |
| AF078694_p1 | Alpha 6 integrin subunit (ITGA6) gene, promoter sequence | −423 |
| AF086788_p1 | Aconitase (ACO2) gene, nuclear gene encoding mitochondrial | −141 |
| | protein, exon 1 | |
| AF086926_p1 | Dynactin 1 (DCTN1) gene, exon 1. | −61 |
| AF088888_p1 | Retinoic acid receptor alpha (RARA) gene, exon 1. | 34 |
| AF092906_p1 | Ribosomal protein S19 (RPS19) gene, exon 1 and 2. | −359 |
| AF092926_p2 | Microsomal glutathione transferase (MGST1) gene, exons 1 and | 384 |
| | 2, alternatively spliced products | |
| AF104233_p1 | ADP-ribosylation factor 4 (ARF4) gene, exon 1. | −670 |
| AF112181_p1 | Interferon regulatory factor 3 (IRF3) gene, promoter region and | 517 |
| | partial cds | |
| AF112229_p1 | CD30 protein (CD30) gene, promoter, exon 1, and partial cds | 229 |
| AF112229_p1 | CD30 protein (CD30) gene, promoter, exon 1, and partial cds | 23 |
| AF112482_p1 | Aldehyde reductase (AKR1A1) gene, exon 1 and promoter | −1622 |
| | sequence | |
| AF126958_p1 | Outer membrane receptor Tom20 (TOM20) gene, exon 1; | −188 |
| | nuclear gene encoding mitochondrial protein | |
| AF128893_p1 | telomerase reverse transcriptase (TERT) | −3929 |
| AF128893_p1 | telomerase reverse transcriptase (TERT) | −179 |
| AF128893_p1 | Telomerase reverse transcriptase (TERT) | 29 |
| AF128893_p1 | Telomerase reverse transcriptase (TERT) | 1842 |
| AF132894_p1 | Cathepsin F (CTSF) gene, complete cds. | −371 |
| AF132894_p1 | Cathepsin F (CTSF) gene, complete cds. | −152 |
| AF132894_p1 | Cathepsin F (CTSF) gene, complete cds. | −80 |
| AF134201_p1 | Prostaglandin E2 receptor EP2 subtype (PTGER2) gene, exon 1 | 1313 |
| AF135372_p1 | Synaptobrevin 2 (VAMP2) gene, complete cds. | −821 |
| AF142779_p1 | BN51 protein gene, promoter region and partial cds. | 233 |
| AF144014 | mdm2 | 175 |
| AF145047_p1 | Heme oxygenase-1 gene, promoter region; and exon 1, partial | −43 |
| | sequence | |
| AF147742_p1 | Myeloid cell differentiation protein (MCL1) gene, promoter and | −587 |
| | complete cds | |
| AF148461_i1 | CLNS1A | intron 1 |
| AF148461 | CLNS1A | intron 1 |
| AF149773_p1 | NOD1 protein (NOD1) gene, exons 1, 2, and 3. | −1287 |
| AF149773_p1 | NOD1 protein (NOD1) gene, exons 1, 2, and 3. | −489 |
| AF156731_p1 | Smad7 gene, promoter region. | −91 |
| AF163763_p1 | Elongation factor 1 A-2 (EF1A-2) gene, complete cds. | −528 |
| AF163763_p1 | Elongation factor 1 A-2 (EF1A-2) gene, complete cds. | 1411 |
| AF163776_p1 | TCF1 gene, partial cds. | −1004 |
| AF166335_p1 | Integrin alpha 6 (ITGA6) gene, exon 1. | −407 |
| AF175325_p1 | Eukaryotic initiation factor 4AI (EIF4A1) gene, partial cds | −295 |
| AF175325_p1 | Eukaryotic initiation factor 4AI (EIF4A1) gene, partial cds | 1125 |
| AF187320 | Transferrin receptor | 559 |
| AF196969_p1 | Phenylalkylamine binding protein gene, complete cds; MG81 | 333 |
| | protein gene, partial cds; putative RNA-binding protein 3 RNP | |
| | gene, complete cds; and MG21 pseudogene, complete sequence | |
| AF198614_p1 | Mcl-1 (MCL-1) and Mcl-1 delta S/TM (MCL-1) genes, | −579 |
| | alternative spliced forms, complete cds | |
| AF207550_p1 | Protein translocase, JM26 protein, UDP-galactose translocator, | −1587 |
| | pim-2 protooncogene homolog pim-2h, and shal-typ potassium | |
| | channel genes, complete cds; JM12 protein an transcription | |
| | factor IGHM enhancer 3 genes, partial cds. | |
| AF208234_p1 | Cystatin B (CSTB) gene, promoter region and complete cds | 11 |
| AF208501_p1 | Uncoupling protein 3 (UCP3) gene, promoter and exon 1. | −1050 |
| AF209746_p1 | Beta tropomyosin (TPM2) gene, exons 1 through 8 and partial | −762 |
| | cds | |
| AF223404 | WNT1 inducible signaling pathway protein 1 (WISP1) gene, | −4602 |
| | promoter and partial cds | |
| AF223404_p1 | WNT1 inducible signaling pathway protein 1 (WISP1) gene, | −1101 |
| | promoter and partial cds | |
| AF224272_p1 | Cyclin dependent kinase 4, promoter region. | −125 |
| AF224272_p1 | Cyclin dependent kinase 4, promoter region. | −198 |
| U37022_p1, | cdk4 | −29 |
| AF224272_p1 | | |
| U37022_p1, | cdk4 | 455 |
| AF224272_p1 | | |
| AF239710_p1 | DNA polymerase delta small subunit (POLD2) gene, exons 1 | 287 |
| | through 11 and complete cds | |
| AF255613_p1 | Teratoma-associated tyrosine kinase (TAPK) gene, exons 1 | 349 |
| | through 6 and partial cds | |
| AF257772 | RNA binding protein MCG10 gene | −2257 |
| AF258623_p1 | ATP binding cassette transporter 1 (ABCA1) gene, promoter | −64 |
| | and exon 1 | |
| AF258674_p1 | MUCDHL (MUCDHL) gene, complete cds, alternatively | −469 |
| | spliced | |
| AF270493 | Id2 | −1590 |
| AF289220 | BCL2L12 | 65 |
| AF293386_p1 | Eukaryotic translation initiation factor 5AII (EIF5A2) gene, | −186 |
| | exons 1, 2, and 3 | |
| AJ001686_p1 | NKG2F gene. | −1322 |
| AJ002311_i1 | Synaptogyrin 2 | intron 1 |
| AJ006239_{—} | QDPR; dihydropteridine reductase | intron 1 |
| AJ009866_p1 | pex3 gene (joined CDS, promoter and exon 1). | −1086 |
| AJ009866_p1 | pex3 gene (joined CDS, promoter and exon 1). | −752 |
| AJ010341_p1 | PISSLRE gene, exons 1, 2, and 3 and joined CDS. | 779 |
| AJ010395_i1 | DKC1 | 680 |
| AJ010395_i1 | DKC1 | 653 |
| AJ011802_p1 | OZF gene exon 1. | −676 |
| AJ011802_p1 | OZF gene exon 1. | 27 |
| AJ012453_p1 | MUC5B gene proximal 5′ flanking region. | 1027 |
| AJ131016_p1 | SCL gene locus. | −147 |
| AJ131757 | olr1 | 2481 |
| AJ131612 | dic | 1749 |
| AJ224639_p1 | Surf-5 and Surf-6 genes. | −368 |
| AJ224639_p1 | Surf-5 and Surf-6 genes. | −219 |
| aj238481_p1 | FBP2 gene fructose-1,6-bisphosphatase 2 | −1450 |
| AJ238482_i1 | FBP2; fructose-1,6-bisphosphatase 2 | 1291 |
| AJ238511_i1 | MVP | intron 1 |
| aj238592 | SLAP (src-like adaptor protein) | −2974 |
| AJ243297_i1 | RET | −837 |
| AJ245489_p1 | GCGR gene for glucagon receptor, promoter I. | −1100 |
| AJ249162 | Enhancer from ISG20 | |
| AJ249275_p2 | Partial MTHFR gene for methylenetetrahydrofolate reductase | −1108 |
| AJ249275_p2 | Partial MTHFR gene for methylenetetrahydrofolate reductase | −797 |
| AJ249275_p2 | Partial MTHFR gene for methylenetetrahydrofolate reductase | −774 |
| AJ250235_i1 | FECH gene for ferrochelatase | 5944 |
| AJ250249_p1 | Partial Mif1/KIAA0025 gene, 5′-upstream region. | −475 |
| AJ250249_p1 | Partial Mif1/KIAA0025 gene, 5′-upstream region. | −168 |
| AJ250915_p1 | p10 gene for chaperonin 10 (Hsp10 protein) and p60 gene for | 232 |
| | chaperonin 60 (Hsp60 protein) | |
| AJ250915_p1 | p10 gene for chaperonin 10 (Hsp10 protein) and p60 gene for | −522 |
| | chaperonin 60 (Hsp60 protein) | |
| AJ272029_p1 | Partial CD30 gene for cytokine receptor CD30 and promoter | 951 |
| | region | |
| AL021154 | Id3 | −2895 |
| AL022312_p2 | ATF4 gene | −86 |
| AL121928_p1 | DNA sequence from clone RP11-18I14, gene bA18I14.4 | −1514 |
| AL133551_p1 | SIRT1 gene (Sir2-like proteins (siruitins) type 1) | −1049 |
| AL133551_p1 | SIRT1 gene (Sir2-like proteins (siruitins) type 1) | −423 |
| D00591_p1 | RCC1 gene, exons 1, 2, 3, 4, 5, 6, 7, 8, 9, 10, 11, 12, 13, 14, | 1055 |
| | complete cds | |
| D13156_p1 | Elafin, complete cds. | 343 |
| D13370_p1 | APX gene encoding APEX nuclease, complete cds. | −9 |
| D14668_p1 | Proteasome HC5, 5′-flanking region. | 47 |
| D17616_p1 | N-acetylgalactosamine 6-sulfate sulfatase (GALNS), exon 1 | −187 |
| D21801_p1 | Proteasome subunit HC8, 5′-flanking region. | −660 |
| D28877_i1 | hnRNP protein A2/B1 | 2218 |
| D38592_i1 | MTH1 gene for 8-oxo-dGTPase | −56 |
| D50030_p1 | Hepatocyte growth factor activator, complete cds | 1873 |
| D63395_p1 | NOTCH4, partial cds. | 870 |
| D63861_p1 | Cyclophilin 40, complete cds. | 499 |
| D63861_p1 | Cyclophilin 40, complete cds. | 949 |
| D85429_p1 | Heat shock protein 40, complete cds. | −1346 |
| D85922_i1 | single-minded 2 | 177 |
| D85922_i1 | single-minded 2 | −360 |
| D87675_p1 | Amyloid precursor protein, complete cds. | 3267 |
| D87675_p1 | Amyloid precursor protein, complete cds. | 568 |
| D87943_i1 | Alpha(1,2)fucosyltransferase | intron 1 |
| D90084_p1 | Pyruvate dehydrogenase(EC 1.2.4.1)alpha subunit gene, exons 1-11 | 826 |
| J00153_p1 | Alpha globin psi-alpha-1, alpha-2 and alpha-1 genes, complete | 371 |
| | cds | |
| J03466_p1 | Insulin receptor gene, exon 1, clone p-lambda EA2. | −876 |
| J03764_p1 | Plasminogen activator inhibitor-1 gene, exons 2 to 9. | −556 |
| J03764_p1 | Plasminogen activator inhibitor-1 gene, exons 2 to 9. | −440 |
| J04038_p1 | Glyceraldehyde-3-phosphate dehydrogenase (GAPDH) gene, | 666 |
| | complete cds | |
| J04111_p1 | c-jun proto oncogene (JUN), complete cds, clone hCJ-1. | 946 |
| J04201_p1 | Beta-polymerase gene, exons 1 and 2. | −525 |
| J04809_p1 | Cytosolic adenylate kinase (AK1) gene, complete cds. | −818 |
| J04988_p1 | 90 kD heat shock protein gene, complete cds. | 374 |
| J05096_p1 | Na, K-ATPase subunit alpha 2 (ATP1A2) gene, complete cds. | 1228 |
| J05253_i1 | Interstitial retinol-binding prot. prec. (IRBP) | 4328 |
| K02402_i1 | Coagulation factor IX | 2866 |
| K03014_p1 | MHC class II HLA-SB-beta-1 gene (untyped), clone LC11. | 1564 |
| K03021_p1 | Tissue plasminogen activator (PLAT) gene, complete cds. | 557 |
| L04147_p1 | Neurofilament light chain (NEFL) gene, promoter region. | −1239 |
| L06162_p1 | Breast cancer-associated antigen (DF3) gene, 5′ end and | −1583 |
| | promoter region | |
| L06484_p1 | Acetylcholinesterase (ACHE) gene, exons 1-2, and promoter | −262 |
| | region | |
| L07485_p1 | Deoxycytidine kinase gene, promoter region. | −450 |
| L10137_p1 | Histone (H2AZ) gene, promoter sequence. | −392 |
| L10347_i1 | Pro-alphal type II collagen (COL2A1) | 1219 |
| L10822_p1 | Gastrin receptor gene, complete cds. | 1872 |
| L11016_p1 | Lymphotoxin-beta gene, complete cds. | −1531 |
| L12399_p1 | Nuclear lamin A and nuclear lamin C gene, exon 1. | −707 |
| L13391_p1 | Helix-loop-helix basic phosphoprotein (G0S8) gene, complete | −823 |
| | cds | |
| L14272_p1 | Prohibitin (PHB) gene, exons 1-7. | −352 |
| L16842_p1 | Ubiquinol cytochrome-c reductase core I protein mRNA, | 883 |
| | complete cds | |
| L19314 | HRY (Hairy, or HHL, HES-1) | −1260 |
| L21905_p1 | Troponin I, slow-twitch isoform(TNNI1) gene, exon 1. | −567 |
| L22298_p1 | Moesin gene. | −335 |
| L24442_p1 | Interferon regulatory factor 2 (IRF2) gene, 5′ flank. | −403 |
| L25932_p1 | Integral nuclear envelope inner membrane protein (LBR) gene, | 1019 |
| | 5′ UTR, exons 1 and 2 | |
| 127148 | Galanin gene, 5′ end. | −2487 |
| L27148_p1 | Galanin gene, 5′ end. | −1383 |
| L27587_i1 | CD79b/Ig beta/B29 | −644 |
| L29530_i1 | CACNL1A1 calcium channel L-type alpha 1 sub. | −129 |
| L29766_i1 | Epoxide hydrolase (EPHX1) | −2207 |
| X77738_p1 | Red cell anion exchanger (EPB3, AE1, Band 3) gene, 3′ region | −940 |
| L39891_i1 | Polycystic kidney disease-associated protein (PKD1) | 10910 |
| L41560_i1 | PCBD; pterin-4a-carbinolamine dehydratase | −4219 |
| L41919_p1 | Candidate tumor suppressor HIC-1 (HIC-1) gene, complete cds | 1338 |
| L44140_p1 | Chromosome X region from filamin (FLN) gene to glucose-6- | 1176 |
| | phosphate dehydrogenase (G6PD) gene, complete cds's | |
| L44140_p3 | Chromosome X region from filamin (FLN) gene to glucose-6- | 470 |
| | phosphate dehydrogenase (G6PD) gene, complete cds's | |
| M10090_p1 | Myoglobin gene, exon 1. | 244 |
| M11166_p1 | Prepro-8-arginine-vasopressin-neurophysin II gene, complete | −152 |
| | cds | |
| M11726_p1 | Pancreatic polypeptide gene, complete cds. | 235 |
| M12036_i1 | HER2 | 625 |
| M13934 | RPS14 | 4100 |
| M19283_p1 | Cytoskeletal gamma-actin gene, complete cds. | −142 |
| M19508_p1 | Myeloperoxidase gene, exons 1-4. | 1029 |
| M19720_p1 | L-myc protein gene, complete cds. | 1238 |
| M20317_p1 | Alpha-galactosidase A gene, exon 1. | −221 |
| M20444_p1 | Nucleus-encoded mitochondrial aldehyde dehydrogenase 2 | −123 |
| | (ALDH2) gene, exon 1 | |
| M25161_p1 | Na,K-ATPase beta subunit (ATP1B) gene, exons 1 and 2. | −1119 |
| M26167_p1 | Platelet factor 4 varation 1 (PF4var1) gene, complete cds. | 314 |
| M27274_p1 | Prostate specific antigen gene, complete cds. | −316 |
| M29186_p1 | Triglyceride lipase gene, exon 1. | −765 |
| M29186_p1 | Triglyceride lipase gene, exon 1. | −279. |
| M30137_p1 | ets2 protein gene, 5′ flank. | −398 |
| M31303_p1 | Oncoprotein 18 (Op18) gene, complete cds. | −39 |
| M31303_p1 | Oncoprotein 18 (Op18) gene, complete cds. | −870 |
| M31303_p1 | Oncoprotein 18 (Op18) gene, complete cds. | −39 |
| M31731_p1 | Chronic lymphatic leukemia protein (bcl-3) gene, exon 1, clone | −731 |
| | cLK2 | |
| M32405_p1 | Homologue of rat insulinoma gene (rig), exons 1-4. | 768 |
| M33132_p1 | Proliferating cell nucleolar protein P120 gene, exons 1-15. | −336 |
| M35425_p1 | Hepatic lipase gene, exon 1. | −311 |
| M35425_p1 | Hepatic lipase gene, exon 1. | −798 |
| M37065_p1 | Glutathione S-transferase (GST-pi) pi gene, 5′-flanking region | −1820 |
| M58602_p1 | Platelet-derived endothelial cell growth factor gene, exons 1 | −442 |
| | through 10 | |
| M58602_p1 | Platelet-derived endothelial cell growth factor gene, exons 1 | 232 |
| | through 10 | |
| M60436_p1 | Poly(ADP-ribose) polymerase gene, 5′ end. | 106 |
| m60556 | Transforming growth factor beta-3 | −2160 |
| M60556_p1 | Transforming growth factor beta-3 gene, 5′ end. | −1046 |
| M60858_p1 | Nucleolin gene, complete cds. | 406 |
| M60858_p1 | Nucleolin gene, complete cds. | 429 |
| M61170_p1 | Polymorphic epithelial mucin (PEM) gene, complete cds. | −1510 |
| M61170_p1 | Polymorphic epithelial mucin (PEM) gene, complete cds. | −1595 |
| M63544 | SLBP (histone stem-loop binding protein) | |
| M64231_p1 | Spermidine synthase gene, complete cds. | 549 |
| M64231_p1 | Spermidine synthase gene, complete cds. | −191 |
| M64280_p1 | Complement receptor 2 (CR2, CD21) gene, promoter region. | −130 |
| M65001_p1 | Intercellular adhesion molecule 1 (ICAM-1) gene, exon 1. | 274 |
| M68882_p1 | Steroid 5-alpha-reductase gene, exon 1. | −497 |
| M74239_p1 | (dx patient) phosphoprotein (p18) gene, exon 1. | −39 |
| M77232_p1 | Ribosomal protein S6 gene, complete cds and flanking regions. | 461 |
| M81834_p1 | Alpha-2 collagen type VI gene, exons 1 and 2. | −1240 |
| M84327_p1 | ADP-ribosylation factor 1 gene, exon 1. | −447 |
| M86181_p1 | Prosaposin (PSAP) gene. | −1447 |
| M87841_p1 | Histone (H10) gene, 5′ region. | −1334 |
| M87843_p1 | Transforming growth factor beta-2 gene, 5′ end. | −49 |
| M87843_p1 | Transforming growth factor beta-2 gene, 5′ end. | 752 |
| M88003_p1 | S-adenosylmethionine decarboxylase (AMD1) gene, exon 1. | −399 |
| M90058_p1 | Serglycin gene, exons 1,2, and 3. | −1703 |
| M91463_p1 | Glucose transporter (GLUT4) gene, complete cds. | 1038 |
| M92444_p1 | Apurinic/apyrimidinic endonuclease (HAP1) gene, complete cds | −33 |
| M94363_p1 | Lamin B2 (LAMB2) gene and ppv1 gene sequence. | 421 |
| M94363_p1 | Lamin B2 (LAMB2) gene and ppv1 gene sequence. | 1434 |
| M94579_p1 | Carboxyl ester lipase (CEL) gene, complete cds. | −353 |
| M95623_p1 | Hydroxymethylbilane synthase gene, complete cds. | −453 |
| M95623_p1 | Hydroxymethylbilane synthase gene, complete cds. | −62 |
| M96264_p1 | Galactose-1-phosphate uridyl transferase (GALT) gene, | −89 |
| | complete cds | |
| M97911_p1 | Wegener's granulomatosis autoantigen proteinase 3 gene, exons | 72 |
| | 1, 2, 3, 4, and 5 | |
| M97911_p1 | Wegener's granulomatosis autoantigen proteinase 3 gene, exons | 1699 |
| | 1, 2, 3, 4, and 5 | |
| M99703_p1 | Class II AP endonuclease (APE) gene, partial CDS. | −395 |
| NT_030106 | fra-1 | 913 |
| NT_030106 | fra-1 | 913 |
| S56449_p1 | (PTMA) prothymosin alpha {5′ region, promoter} [human, | −1170 |
| | Genomic, 2025 nt] | |
| S74230_p1 | E2F1 | −1366 |
| S74230_p1 | E2F1 | −364 |
| U00239_p1 | GPAT and AIRC genes promoter sequence. | −189 |
| U01317_p1 | Beta globin region on chromosome 11. | −1295 |
| U02509_p1 | Adenomatous polyposis coli (APC) gene, promoter sequence. | −48 |
| U02509_p1 | Adenomatous polyposis coli (APC) gene, promoter sequence. | −48 |
| U03019_p1 | Melanoma growth stimulatory activity beta (MGSA beta) gene, | −590 |
| | partial cds | |
| U03254_p1 | Acyl-CoA oxidase (AOX) gene, exon 1. | −379 |
| U03735_p1 | MAGE-3 antigen (MAGE-3) gene, complete cds. | 462 |
| U06078_p1 | N-acetylgalactosamine 6-sulphatase (GALNS) gene, exon 1. | −243 |
| U07172_p1 | Insulinoma-associated (IA-1) gene, partial cds. | −314 |
| U07663_p1 | HB9 homeobox gene, exon 1. | −935 |
| U07663_p1 | HB9 homeobox gene, exon 1. | −474 |
| U07802_p1 | Tis11d gene, complete cds. | −565 |
| U07802_p1 | Tis11d gene, complete cds. | 601 |
| U07802_p1 | Tis11d gene, complete cds. | 1280 |
| U08198_p1 | Complement C8 gamma subunit precursor (C8G) gene, | 862 |
| | complete cds | |
| U09360_p1 | Intercellular adhesion molecule-1 gene, promoter region. | −669 |
| U11239_p1 | Cytosolic phospholipase A2 gene, promoter and exon 1. | 457 |
| U14939_p1 | Folylpolyglutamate synthetase gene, partial cds. | 221 |
| U16271_p1 | AMP deaminase isoform L (AMPD2) gene, exons 1A and 1B. | −820 |
| U16271_p1 | AMP deaminase isoform L (AMPD2) gene, exons 1A and 1B. | −587 |
| U16271_p1 | AMP deaminase isoform L (AMPD2) gene, exons 1A and 1B. | 960 |
| U17193_p1 | Bax gene, 5′ region. | 203 |
| U17193_p1 | Bax gene, 5′ region. | 248 |
| U19152_p1 | P protein (P) gene, exon 1. | 97 |
| U19152_p1 | P protein (P) gene, exon 1. | 97 |
| U20499_p1 | Thermolabile phenol sulfotransferase (stm) gene, complete cds | 687 |
| U20734_p1 | Transcription factor junB (junB) gene, 5′ region and complete | 741 |
| | cds | |
| U20734_p1 | Transcription factor junB (junB) gene, 5′ region and complete | 2653 |
| | cds | |
| U21051_p1 | G protein-coupled receptor (GPR4) gene, complete cds. | 1314 |
| U22364_p1 | Cyclin B1 gene, promoter region. | −308 |
| U25822 | Heat shock protein 90 alpha | |
| U27317_p1 | 11 beta-hydroxysteroid dehydrogenase 2 (HSD11B2) gene, | −1256 |
| | complete cds | |
| U27317_p1 | 11 beta-hydroxysteroid dehydrogenase 2 (HSD11B2) gene, | −571 |
| | complete cds | |
| U28054_p1 | Hepatocyte growth factor-like protein homolog gene, complete | −1725 |
| | cds | |
| U29201 | Metastasis control (nm23-H1) gene, 5′-region. | 260 |
| U29201_p1 | Metastasis control (nm23-H1) gene, 5′-region. | 11 |
| U29927_p1 | AMP deaminase (AMPD3) gene, exon 1b and 1c and promoter | −975 |
| U30787_p1 | Uroporphyrinogen decarboxylase (URO-D) gene, complete cds. | 925 |
| U31120_p1 | Interleukin-13 (IL-13) precursor gene, complete cds. | 671 |
| U32323_p1 | Interleukin-11 receptor alpha chain gene, complete cds. | −358 |
| U33446_p1 | Prostasin gene, complete cds. | −368 |
| U33453_p1 | Protease nexin-1 (PN1) gene, promoter region. | −286 |
| U33453_p1 | Protease nexin-1 (PN1) gene, promoter region. | −1004 |
| U33947_p1 | Medium chain acyl CoA dehydrogenase (MCAD) gene, | −1312 |
| | promoter region | |
| U34070_p1 | CCAAT/enhancer binding protein alpha gene, complete cds. | −268 |
| U34859_p1 | Tyrosine kinase (blk) gene, 5′ flanking region and exon 1 | −1242 |
| U35052_p1 | Helix-loop-helix protein (HEB) gene promoter region and exon 1 | −790 |
| U37106_p1 | Erythroid Kruppel-like factor EKLF gene, complete cds. | 1129 |
| U37106_p1 | Erythroid Kruppel-like factor EKLF gene, complete cds. | 1812 |
| U41448_p1 | Ribosomal protein S26 (RPS26) gene, complete cds. | 330 |
| U43140_p1 | RhoG (ARH-G) gene, promoter region. | −848 |
| U43748_p1 | Frataxin (FRDA) gene, promoter region and exon 1. | 1535 |
| U47284 | Cyclin D2 | −1329 |
| U47284 | Cyclin D2 | −1158 |
| U47289_p1 | Spasmolytic polypeptide (SP) gene, 5′ region and exon 1. | −291 |
| U50871_p1 | Familial Alzheimer's disease (STM2) gene, complete cds. | −225 |
| U50871_p1 | Familial Alzheimer's disease (STM2) gene, complete cds. | −74 |
| U52428_p1 | Fatty acid synthase gene, partial cds. | −357 |
| U52428_p1 | Fatty acid synthase gene, partial cds. | −913 |
| U55231_p1 | Melanoma tumor antigen (Mart-1) promoter region | −714 |
| U52694_p1 | Creb-rp gene, exon land partial cds. | −94 |
| U56438_p1 | Dioxin-inducible cytochrome P450 (CYP1B1) gene, complete | −1680 |
| | cds. | |
| U63108_p1 | Eukaryotic initiation factor 4E (eIF4E) gene, promoter region | −75 |
| | and partial cds | |
| U63630 | MCM4 and DNA-PKcs (pos relative to MCM4) | 820 |
| U63721_p1 | Elastin (ELN) gene, partial cds, and LIM-kinase (LIMK1) gene; | −875 |
| | complete cds | |
| U63721_p1 | Elastin (ELN) gene, partial cds, and LIM-kinase (LIMK1) gene, | −883 |
| | complete cds | |
| U63833_p1 | PAX6 gene, promoter region and exons 1 and 2. | −822 |
| U64864_p1 | PD-1 gene, promoter region and partial cds. | −68 |
| U68093_p1 | Poly(A)-binding protein (PABP) gene, promoter region and | −376 |
| | exon 1 | |
| U71187_p1 | Cholesteryl ester transfer protein (CETP) gene, partial cds and | −1014 |
| | promoter region | |
| U72648_p1 | Alpha2-C4-adrenergic receptor gene, complete cds. | −566 |
| U73167 | Semaphorin V | 6744 |
| U73167 | SM15 gene for IFRD2, interferon-related (PC4, TIS7 | 199 |
| | homologue) | |
| U73167 | SM15 gene for IFRD2, interferon-related (PC4, TIS7 | 225 |
| | homologue) | |
| U75285 | Survivin | 7796 |
| U76667_p1 | Anion exchanger 2 (SLC4A2) gene, alternative promoters b1 | −472 |
| | and b2, exons 2, 1b1, 1b2 and 3 to 8 | |
| U76667_p1 | Anion exchanger 2 (SLC4A2) gene, alternative promoters b1 | −613 |
| | and b2, exons 2, 1b1, 1b2 and 3 to 8 | |
| U76667_p1 | Anion exchanger 2 (SLC4A2) gene, alternative promoters b1 | −580 |
| | and b2, exons 2, 1b1, 1b2 and 3 to 8 | |
| U62290_p1 | AE2 anion exchanger (SLC4A2) gene, promoter region and | −743 |
| | exon 1, complete sequence | |
| U77629_p1 | Achaete-Scute homologue 2 (ASCL2) gene, complete cds. | 562 |
| U78027_p1 | Bruton's tyrosine kinase (BTK), alpha-D-galactosidase A | 176 |
| | (GLA), L44-like ribosomal protein (L44L) and FTP3 (FTP3) | |
| | genes complete cds | |
| U78027_p1 | Bruton's tyrosine kinase (BTK), alpha-D-galactosidase A | 483 |
| | (GLA), L44-like ribosomal protein (L44L) and FTP3 (FTP3) | |
| | genes complete cds | |
| U78027_p3 | Bruton's tyrosine kinase (BTK), alpha-D-galactosidase A | −254 |
| | (GLA), L44-like ribosomal protein (L44L) and FTP3 (FTP3) | |
| | genes complete cds | |
| U78027_p3 | Bruton's tyrosine kinase (BTK), alpha-D-galactosidase A | −221 |
| | (GLA), L44-like ribosomal protein (L44L) and FTP3 (FTP3) | |
| | genes complete cds | |
| U78073_p1 | Polo like kinase (PLK) gene, promoter and partial cds. | −160 |
| U79410_p1 | Thrombospondin 2 (THBS2) gene, promoter region and exons | 650 |
| | 1A and 1B | |
| u82618 | Aspartylglucosaminidase promoter | promoter |
| U86758_p1 | Netrin-2 like protein (NTN2L) gene, complete cds. | 374 |
| U86758_p1 | Netrin-2 like protein (NTN2L) gene, complete cds. | −1446 |
| U86758_p1 | Netrin-2 like protein (NTN2L) gene, complete cds. | −521 |
| U87926_p1 | Aconitate hydratase (ACO2) gene, exon 1. | −139 |
| U88317_p1 | 15-lipoxygenase gene, complete cds. | −295 |
| U89323_p1 | Interleukin-12 p40 gene, exon 1, complete sequence. | −955 |
| U92868_p1 | Reduced folate carrier (RFC1) gene, exons 1a, 1c and 1b | 11 |
| U93237_p1 | Menin (MEN1) gene, complete cds. | −369 |
| u96876 | INSIG1 (insulin induced protein 1) | 3218 |
| V00488_p1 | Alpha-globin germ line gene. | 371 |
| X03493_p1 | c-sis proto-oncogene 5′ region. | −252 |
| X83706_p1 | c-sis proto-oncogene (alternative exon-1a). | −208 |
| X02158_p1 | Erythropoietin. | 382 |
| X05006_p1 | S-protein gene, complete cds. (Vitronectin) | −1097 |
| AC011462, | TGF beta gene (from AC011462, J04431, X05839) | −1018 |
| J04431, X05839 | | |
| AC011462, | TGF beta gene (from AC011462, J04431, X05839) | 2201 |
| J04431, X05839 | | |
| AC011462, | TGF beta gene (from AC011462, J04431, X05839) | 2779 |
| J04431, X05839 | | |
| X07056_p1 | Alpha-amylase (EC 3.2.1.1) gene AMY2A 5-flank and exon 1. | −669 |
| X07871_p1 | CD2 gene exons 1 and 2 (and joined CDS). | −33 |
| X12671_p1 | Heterogeneous nuclear ribonucleoprotein (hnRNP) core protein | −442 |
| | A1 | |
| X12671_p1 | Heterogeneous nuclear ribonucleoprotein (hnRNP) core protein | −379 |
| | A1 | |
| X12671_p1 | Heterogeneous nuclear ribonucleoprotein (hnRNP) core protein | 553 |
| | A1 | |
| X13546_p1 | HMG-17 gene for non-histone chromosomal protein HMG-17. | −104 |
| X14940_p1 | C mu gene for IgM heavy chain exons CH1-4, secretory. | −1225 |
| x15723 | Furin gene, exons 1 through 8. | −2326 |
| X16287_p1 | Alpha-enolase gene for non-neuronal enolase (EC 4.2.1.11) | −796 |
| | exon 1 (and joined mRNA) | |
| x51898 | bc1-2 gene 5′-flanking region | promoter |
| X51952_p1 | UCP gene for uncoupling protein exons 1 and 2. | −259 |
| X52601_p1 | hTOP1 gene for topoisomerase, 5′end. | −358 |
| X54816_p1 | Alpha-1-microglobulin-bikunin, exons 1-5 (encoding alpha-1- | −563 |
| | microglobulin, N-terminus.) | |
| X56997_p1 | UbA52 gene coding for ubiquitin-52 amino acid fusion protein. | −145 |
| X57928_p1 | Prostatic secretory protein PSP-94, exon 1 and joined CDS | −294 |
| X59964_p1 | CST4 gene for Cystatin D. | −373 |
| X60482_p1 | H4/b gene for H4 histone. | 452 |
| X60484_p1 | H4/e gene for H4 histone. | 524 |
| X61282_p1 | N-RAS promoter region. | −46 |
| X62654_p1 | Me491/CD63 antigen. | −178 |
| x62668 | Melanotransferrin gene enhancer | |
| X67123_p1 | TRAP gene for tartrate-resistant acid phosphatase type 5, exons | −144 |
| | 1-4 | |
| X68969_p1 | Betal integrin. | −940 |
| X69118_p1 | Promoter sequence of MUC1 gene. | −1570 |
| X70286_p1 | Thioredoxin, exon 1. | −423 |
| X72735_p1 | Arylsulfatase B, exon 1. | −868 |
| X74107_p1 | Antigen of the monoclonal antibody Ki-67. | 1578 |
| X74840_p1 | Wilms tumor (WT1) gene promoter. | 213 |
| x74961 | ACPP gene for prostatic acid phosphatase | −2034 |
| X77491_p1 | G11 exon 5, 6, 7 and gene for C4A. | −77 |
| X82032_p1 | B-myb gene. | −309 |
| X82201_p1 | RPL19 gene. | −261 |
| X82245_p1 | Nidogen gene (exon 1). | −1893 |
| X82245_p1 | Nidogen gene (exon 1). | 1138 |
| X87344_p14 | DMB gene | −1988 |
| X87344_p15 | DMB gene | −1178 |
| X94359_p1 | 5′ region of angiotensin-I converting enzyme. | −1177 |
| X94359_p1 | 5′ region of angiotensin-I converting enzyme. | −861 |
| X94563_p1 | Dbi/acbp gene exon 1 & 2. | −123 |
| X95151_p1 | Brca2 gene exon 1. | −19 |
| X95536_p1 | Ear1 gene. | −471 |
| X98053_p1 | G13 gene. | −109 |
| Y00067_p1 | Neurofilament subunit M (NF-M). | −134 |
| Y00371_p1 | Hsc70 gene for 71 kd heat shock cognate protein. | 1666 |
| Y00371_p1 | Hsc70 gene for 71 kd heat shock cognate protein. | 558 |
| Y07558_p1 | PILOT gene, 5′ flanking region. | −846 |
| Y07712_p1 | ATFa gene, promoter region. | −201 |
| Y08733_p1 | MIIX gene, promoter region. | −924 |
| y09540 | AHSG gene, partial. | −3072 |
| Y10284_p1 | TRAF1 gene, putative promoter region. | −485 |
| Y12773_p1 | TRIDENT/HFH11 gene, promoter sequence. | −113 |
| Y13901_p1 | FGFR-4 gene. | 629 |
| Y15082_p1 | p14.5 gene, partial exon 1 and promoter; hPOP1 gene exon 1 | −167 |
| | and promoter and bidirectional promoter region | |
| Z19561_p1 | FAH gene promoter region. | −554 |
| Z21818_p1 | Carcinoembryonic antigen gene. | −41 |
| Z23091_p1 | GPV gene encoding platelet glycoprotein V precursor. | −787 |
| Z26491_p1 | Catechol O-methyltransferase. | 34 |
| Z29077_p1 | Cdc25 gene promoter region. | −659 |
| Z29078_p1 | Cyclin D1 gene promoter region. | −552 |
| Z84811_p1 | Muscle nicotinic acetylcholine receptor gene promoter. | −740 |

Table 2 discloses nucleic acids, genes, or polypeptides from Table 1, for use in the claimed methods.

TABLE 2

| Accession number | Gene definition |
|---|---|
| AF009247_p1 | CLCN6 |
| AF148461_i1; NP_001284; P54105 | CLNS1A |
| u76667_p1 | Anion exchanger 2 (SLC4A2) |
| AF255613_p1 | Teratoma-associated tyrosine |
| | kinase (TAPK) (or gklp) |
| U86758_p1 | Netrin-2 like protein (NTN2L) |
| | gene, complete cds. |

III. Computer Search for Genomic Sites for, Use in ChIP Assays.

Computer searches were performed according to Wang, et al. (2001) J. Biol. Chem. 276:43604-43610. The search encompassed a population of 6541 Genbank entries. Human genomic sequences were searched for an E box motif, located with a distance of 2 kb on either side of transcription start sites (Abstract of Mol, et al. (1995) Mol. Cell. Biol. 15:6999-7009). 1630 loci scored positive, with one or more E-boxes within the predetermined boundaries, identifying 2224 E-boxes. 93 additional loci were included in the screen, of which 69 E-boxes were outside the +/−2 kb boundary.

IV. ChIP Assays.

ChIP assays were performed as described in Frank, et al. (2001) supra, with the following modifications. Fixed cells (1.5-3.3×10⁸ cells) were sonicated in 6 ml of SDS buffer. The lysate was diluted with 3 ml of Triton Dilution Buffer (100 mM Tris, pH 8.6, 100 mM NaCl, 5 mM EDTA, 5.0% Triton® X-100). Immunoprecipitation was performed using 9 ml of lysate, and either 0.05 mg polyclonal anti-c-Myc antibody N-262 (cat. no. SC764; Santa Cruz Biotechnology, Santa Cruz, Calif.) or 0.5 ml of blocked protein A beads, i.e., a 50% slurry of Protein A-Sepharose® (Amersham Biosciences, Piscataway, N.J.), per sample. For large scale experiments, DNA preparations from three independent ChIPs were pooled and diluted in 6 ml of water.

Polymerase chain reactions (PCR) were performed with 0.004 ml of DNA and 800 nM primers, diluted in a final volume of 0.02 ml in SYBR Green Reaction Mix (Applied Biosystems, Foster City, Calif.). SYBR 0 Green PCR Core Reagents (Applied Biosystems, Foster City, Calif.) was used to monitor PCR product.

Control immunoprecipitations were performed in a variety of ways, e.g. by using pre-immune serum rather than by using anti-Myc antibody, or by using Myc-deficient cells with the standard ChIP procedure.

ChIP assays on a large number of E box target sites were conducted on chromatin from five different cell lines, U-937 cells, HL60 cells, P493-6 cells, T98G cells, and WSI cells. Chromatin from U-937 cells were used for two types of tests, CHIP assays where the targets were E boxes associated with a promoter, and ChIP assays where the 134 E boxes were randomly chosen from chromosome 21, i.e., not necessarily associated with a promoter. Chromatin from the other cell lines were subjected to ChIP assays targeting only FS boxes associated with a promoter.

The results from the U937 cells were as follows. 809 E-boxes/genes were selected from a list of 2224 E-boxes for usein ChIP assays. A computer screen of U937 cells and HL60 cells identified 351 promoter-associated sites, and these sites were used in ChIP assays of U937 cells. An additional 458 sites were tested in U937 cells, where these additional sites were selected according to biological interest, resulting in a total of 809 sites tested in the U937 cells. ChIP assays were applied to these 809 target sites were conducted with anti-Myc antibody (experimentals) and without anti-Myc antibody (controls). Myc bound to 336 (42%) of the 809 sites, i.e., there were about 340 positive screening sites. Recovery for the control assays ranged from about 0.01% input to about 0.06% input, while recovery for the experimental assays yielded recovery data ranging from a recovery of about 0.02% input to a recovery of about 2.0% input. The positive-screening sites included E-boxes/genes from, e.g., NUC, HSP10/60, CAD, TERT, GPAT/AIRC, and cyclin D2.

U937 cells were also used for a separate study that served as a control study. A number of randomly occurring E-boxes were analyzed, that is, E-boxes not necessarily associated with promoters. 134 Randomly occurring E-boxes in chromosome 21 of U-937 cells were subjected to ChIP assays. Myc bound to five of these sites (3.7%) at relatively low levels though none screened positive by the above-stated criteria. None of the target E-boxes were bound at high levels. In most cases, recovery of the targeted gene for both control and experimental ChIP immunoprecipitations ranged from only about 0.01% to only about 0.08%.

HL60 cell results were as follows. In studies with chromatin from HL60 cells, 125 (36%) of the 351 promoter-associated E-boxes/genes tested screened positive in the ChIP assays.

P493-6 cell line results were as follows. These cells allow repression of a c-Myc transgene by tetracycline (Tet), resulting in G1 arrest in the presence of serum. Subsequent removal of tetracycline induces Myc, and re-entry into the cell cycle (Schuhmacher, et al. (1999) Curr. Biol. 9:1255-1258; Schuhmacher, et al. (2001) Nucl. Acids Res. 29:397-406). The sources of cells in the following ChIP assays were Tet-treated cell preparations, where Myc was repressed, and Tet-removed-cell preparations to allow induction of Myc (culture for 8 h after removal of Tet).

ChIP immunoprecipitation assays on the same collection of target E boxes/genes were conducted under three different conditions: (1) With anti-Myc antibody (no Tet); (2) With anti-Myc antibody (plus Tet); and (3) Control without anti-Myc antibody (no Tet).

The highest signals were from ChIP assays using anti-Myc antibodies, where cells had been induced to synthesize Myc. The recovery for most of the E boxes/genes in this test was above 0.1% input. Intermediate results were produced by ChIP assays containing anti-Myc antibodies, performed on non-induced cells. The recovery for most of the E/boxes/genes in this test was below 0.1% input. Control ChIP assays without anti-Myc antibody using non-induced cells showed relatively low signals. The recovery for most of the E boxes/genes in this test was below 0.04% input. Upon induction of Myc, 330 of the 388 (85%) tested E-boxes/genes tested positive. Since this enrichment was dependent on removal of tetracycline, it demonstrated that a positive signal in the ChIP assay was dependent on increased concentrations of intracellular Myc protein.

Human glioblastoma cells (T98G) were studied. This cell line was pre-treated for four hours with serum before use in ChIP assays. Experimental assay mixtures containing anti-Myc antibody and control assay mixtures without anti-Myc antibody were assembled, and recovery for each E-box/gene was expressed as percent input. The ChiP assay signal was greater for the experimental ChIP assays than for the control ChIP assays, for nearly all of the E boxes/genes tested. Recovery of the target genes, for most of the experimental ChIP assays, X ranged from about 0.008% input to about 0.8% input, while the corresponding values for control ChIP assays ranged from only about 0.001% input to only about 0.02% input.

Primary human fibroblasts (WS1) were studied. The cell line was pre-treated for four hours with serum before use in ChIP assays. Experimental chIP assays containing anti-Myc antibody and control ChIP assays without anti-Myc antibody were conducted. Recovery for each E-box/gene was expressed as percent input for the experimental and control assays. The results demonstrated that Myc binding resulted in a signal-above control for about half of the genes tested in serum-treated WS1 cells.

Following collection of the ChIP data from the various cell lines, the data from specific pairs of different cell lines were compared to each other, e.g., by comparing results from a particular E box/gene from HL60 cells with the results from that same gene from U937 cells. The pairwise comparisons were made for all of the E boxes/genes tested that were common to both cell lines.

The goal was to determine if Myc bound to overlapping populations of target sites in various cell lines. The comparisons, i.e., pairwise plots, disclosed ChIP data from U937 cells versus from HL60 cells; U937 cells versus P493-6 cells; T98G cells versus U937 cells; and WSI cells versus U937 cells. In all combinations, most of the high-affinity sites clustered together, as did the low affinity sites, resulting in a roughly linear continuum. In other words, the ChIP signal (% input), for any given gene, was roughly comparable in tests among the different cell lines. As stated above, the resulting plots were roughly linear. Thus, the relative Myc-binding efficiencies of promoter E-boxes was conserved among different cell lines.

In all pairwise combinations, there were a minority of outliers, that is, sites that were bound efficiently only in a given cell line. These differences might be due to tissue-specific accessibility of chromatin or to exclusion of Myc binding through de novo methylation of selected CpG islands.

V. Cellular Levels of Myc Protein.

Myc protein was measured in HL60, U937 cells, Raji cells (Raji Burkitt lymphoma), P493-6 cells, T98Q cells, and WS1 cells using western blot analysis. For each blot, 50 micrograms of whole cell lysate from exponentially growing cells was separated by SDS PAGE and probed using a monoclonal anti-Myc antibody (9E10). HL-60 and WS1 cells expressed low levels of Myc, while Raji cells and induced P496-3 cells, contained relatively high levels of Myc. U-937 cells and T98G cells expressed intermediate amounts of Myc protein, where expression by U-937 cells was greater than for the T98 cells. The range of Myc protein levels in these cell lines covered about two orders of magnitude.

Myc was also measured in P496-3 cells over the course of time, with induction of Myc by removal of tetracycline (Tet) and measurement at 0, 1, 3, 6, 9, and 12 h after induction by Tet removal. The results demonstrated no detectable expression at t=0 h, slight expression at t=1 h, with about 50% maximal expression occurring at 3 h and 6 h, and maximal expression at 9 h and 12 h.

These results, taken with those, comparing ChIP signals acquired from tests of the different cell lines, demonstrate that the distribution of binding to various Myc targets remains approximately the same, even where intracellular levels of Myc differ widely.

VI. Correlations Between Myc-Binding and Induction of mRNA Expression.

Time courses for induction of a number of genes in P493-6 cells and in T98G cells were studied, where induction was by Myc-induction (P493-6 cells) or by serum induction (T98 cells). mRNA encoding rpP0, NUC, DKc1, CASP8, AMPD2, and C-MET was measured at t=0, 1, 3, 6, 9, and 12 hours, in both types of cells.

With P493-6 cells, maximal or near-maximal induction of NUC (7-fold induction) and DKC1 (6-fold) appeared at 6-12 h after Tet removal. Little or no increase in mRNA levels were detected for message expressed from fpPO, CASP8, AMPD2, or c-MET genes. In all cases, little or no change in gene expression was found in control incubations where continued presence of Tet prevented induction of Myc.

With T98G cells, maximal induction of NUC (6-fold), DKC1 (14-fold), and C-MET (10-fold) occurred at about 9 hours. RpPO was gradually induced, over the course of six hours, to a maximum of about 2.5-fold, where maximal induction was found at 6-12 hours. There was little or no detectable induction of CASP8 and AMPD2 during the 12 hour incubation period.

The possible correlation between ChiP assay % input and fold-induction of mRNA expression was studied in P493-6 cells, where 75 genes were examined, and for T98 cells, where 37 genes were examined. Although there was a correlation between ChIP signal and fold-mRNA induction for some genes, there was little overall correlation between ChIP signals and fold-mRNA induction for the genes that were tested. Thus, for some genes Myc binding alone can be sufficient to provoke increases in gene expression, while for other genes factors in addition to Myc binding may be required from gene expression.

VII. Conditions of Cell Culture.

U937 and HL60 cells were grown in RPMI (Roswell Park Memorial Institute) medium supplemented with 10% fetal calf serum. For analysis by the ChIP technique, 1.5 liters of exponentially growing cells were diluted to 2-3×10⁵ cells/ml one day before harvesting. P-496-3 cells are described (Kempkes, et al. (1995) EMBO J. 14:8896). P496-3 cells were grown in RPMI medium supplemented with 10% fetal calf serum, NEAA (BioWhittaker, Inc., Walkersville, Md.), and 2 mM L-glutamine (BioWhittaker, Inc.). Repression and re-expression of Myc was according to Schuhmacher, et al. (2001) Nucl. Acids Res. 29:397-406. For ChIP, 2 liters of exponentially growing cells were diluted to 3×10⁵ cells/ml and tetracycline (0.0001 mg/ml) (Sigma-Aldrich, St. Louis, Mo.) was added for 72 h. To re-induce expression of Myc, cells were washed three times in prewarmed RPMI medium containing 10% fetal calf serum before culturing for the indicated period of time.

T98G and WS1 were from American Type Culture Collection (Manassas, Va.) and grown in D-MEM supplemented with 10% fetal calf serum. Cells were rendered quiescent by growth to confluent density, followed by incubation for three days in serum-free medium. To induce cell cycle entry, cells were harvested by trypsinization and re-seeded 1:4 onto plates containing D-MEM/10% FCS. For ChIP assays, cells from 15 confluent 150 mm dishes, or the equivalent amount of cells, following dilution (splitting) were used. One confluent plate, or the equivalent amount of cells, were used for RNA extraction.

Many modifications and variations of this invention, as will be apparent to one of ordinary skill in the art can be made to adapt to a particular situation, material, composition of matter, process, process step or steps, to preserve the objective, spirit, and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of the equivalents to which such claims are entitled; and the invention is not to be limited by the specific embodiments that have been presented herein by way of example.

What is claimed is:

1. A method of regulating cell proliferation comprising modulating the activity of a gene or polypeptide of Table 2.

2. The method of claim 1, wherein the gene is positive for Myc binding in a chromatin immunoprecipitation (CHIP) assay.

3. The method of claim 1, wherein the modulating is inhibiting.

4. The method of claim 1, wherein the modulating is activating.

5. The method of claim 1, wherein the cell proliferation is oncogenic.

6. The method of claim 1, wherein the modulating is by a binding composition.

7. The method of claim 6, wherein the binding composition comprises an antigen-binding site of an antibody, a soluble receptor, a nucleic acid, or a small molecule.

8. The method of claim 7, wherein the binding composition comprises:
a) a human or humanized antibody;
b) a monoclonal antibody;
c) a polyclonal antibody;
d) an Fab fragment or an F(ab′)₂ fragment;
e) a peptide mimetic of an antibody;
f) a detectable label; or
g) an anti-sense nucleic acid.

9. A method for the diagnosis of a proliferative condition comprising detecting or determining the expression or activity of at least one gene or polypeptide of Table 2.

10. The method of claim 9, wherein the gene is positive for Myc binding in a ChIP assay.

11. The method of claim 9, wherein the detecting or determining is by a binding composition comprising the antigen binding site from an antibody, a soluble receptor, or a nucleic acid.

12. The method of claim 11, wherein the binding composition comprises:
a) a human or humanized antibody;
b) a monoclonal antibody;
c) a polyclonal antibody;
d) an Fab fragment or an F(ab′)₂ fragment;
e) a peptide mimetic of an antibody;
f) a nucleic acid probe or nucleic acid primer; or
g) a detectable label.

13. A method of treating a subject suffering from a proliferative disorder comprising administering to the subject an effective amount of an agonist or antagonist of at least one gene or polypeptide of Table 2.

14. The method of claim 13, wherein the gene is positive for Myc binding in a ChIP assay.

15. The method of claim 13, wherein the proliferative disorder is oncogenic.

16. The method of claim 13, wherein the treating is by a binding composition.

17. The method of claim 16, wherein the binding composition comprises an antigen-binding site of an antibody, a soluble receptor, a nucleic acid, or a small molecule.

18. The method of claim 17, wherein the binding composition comprises:
a) a human or humanized antibody;
b) a monoclonal antibody;
c) a polyclonal antibody;
d) an Fab fragment organ F(ab′)₂ fragment;
e) a peptide mimetic of an antibody;
f) a detectable label; or
g) an anti-sense nucleic acid
